SEQUENCE LISTING (21 sequences)

SEQ ID NO: 1 - protein, 431 aa, Capnocytophaga canimorsus
QTENTTEQRLNAWFTDAKLGIFIHWGYYGVNGITESWSMYHKRISHTDYLKQGQKFTASNYNPDQWLDLFERVGAKYVVLTTKHHDGVALWNTKYSNLSTVKHASARKDVLTPFVEAVRKKNIKLGLYYSLCDWSHPDYQPVTFQRRDVRKLYPQKGQQKMTPWERFTAFNFNQLEELSQFKPDLWWFDGDWEHKPEEWKSKEIKEKLLFWNPQTVVNSRLNQYGDYKTPEQGVPVVRPEGAWEFCMTMNDNWGYFPTDTNYKPIAQIIRTFVEVIGSGGNLLLNVGPKPDGTIPQEQVLRLEALGNWISKHKTAVFDTQAGLPYGHFFGPTLLSKDKTKIYLCLFDNPKNYILLKGIQNKVKSIKVLGAEQKISFERNGGAAWNNIPGILRISVPADQSLDPYVTVLEVHLEGELSLYRGHGNAVELNMV

SEQ ID NO: 2 - protein, 449 aa, Omnitrophica bacterium OLB16
MRYILAVLLMVGMMAGAATAVTYEPTWESLDSRPNPAWFDEAKFGIFIHWGVYAVPAWGSKGKYSEWYWNDMMDPNGETWKFHLKTYGEDFKYQDFAPMFKAEMFDPAQWADIFARSGAKYVVLTSKHHEGFCLWPSPDSWNWNSVDIGPHRDLCGDLTQAVRDRGLKMGFYYSLYEWFNPIYKTDVHRYVDQHMLPQLKDLVNRYQPSLIFSDGEWDHPSDVWRSTEFLAWLYNESPSREDVIVDDRWGKDTRGHHGGYYTTEYGNIYQAPEDAFQKRKWEECRGMGASFGYNRNETIDEYKPAGELIHLLIELVARGGNLLLDIGPTADGRIPVIMQQRLLEIGDWLKENGEGIYGSSPWRVNAEGDSVRYTTRDGAVYAHLLKWPGAELALESPKAGGTVEASLLGWPEPLACKVENGKIHISMPVIPPDNNTIRHAFVIRLKGVE

SEQ ID NO: 3 - protein, 414 aa, Lactobacillus casei
TEPLPRIQHYEDLGLGLFIHWGLYSQMAVGEWTELIHHRNQHDYEQLIKTFTAAQFDAKKIAHAAKAVGAKYIVLTTKHHEGFFLYDTKGLSDFDVMHAPARRDLIAEFVAACREEDLLPFFYMATYDWHTPLYDDDFPAYLTYLQKSVEVLCRNYGPVGGFWFDGNWNKKDADWHLPELYGMIRHYQPNAIIVNNTGLKNRGQVSDPEIDVVTYERRTPDEIYHGAPNEKYVAGEISITLNQHWGIAANDLNYKSPAEVIETVAHARHIGANILVNIGLTGTGAIPAAAQTYMHLLGRWTAMAAPVLYKGRPVPVTSAHGTRDFVLHTSKHDFLCILDLQVVGNDNVVLGGEGVNPRSFVGIGQPIQRIHWLDNDEVLSFTQDLDKKVLTVDATGYPYGSDWVVRIAQIDYEV

SEQ ID NO: 4 - protein, 427 aa, Chitinophaga pinensis
QQHTTADSIREKMQWFADAKLGIFIHWGIYSVNGVDESWSFHNRKISYTDYMQQLKGFTAKNYHPEEWAALIKESGARYAVMTTKHHDGVALWDSKLSKLDIANSTPAKRDVLTPLYNALRQQGIKAGAYFSLIDWSYPDYPQFLKDSSRYDAKKDPARWQRFLNFYEGQLKEVMQQYNPDLWWFDGDWEHSAEEWEAVKIRKMLTGHNPNTIINGRLQAYGDYDTPEQNFPVSRPHYNWWELCMTINNNWGYHPDDTNFKTPYEVITIFADAVSNGGNMLLDIGPAEDGTIPAEEVNVLKELGAWNKKHAPAIFNTVAGIPAGHFYGPTTLSKDSTTLYLFLAGNVNGQVMVKGLVNNIKKISVSGNGQQLTHKVVGKISWSAVPGLVYIDVPANVQDKYMTVLALELDGKIKLYRGKGGFLTNEV

SEQ ID NO: 5 - protein, 429 aa, Streptomyces bingchenggensis
TIPDWFGEAAFGLFVHWDHASQQGVEIGWPLVGHSILPGRTEPEADITVAQYQSTAATFDPHRWDPRAVARLARAAGARYVVFTVRHHAGYSMYHTEHSDFSIAASPYGRDITREFFDALRAEGLRVGVYYSLLDWNHPDYPAFTDADRPYPHDRYRRPSPERWARYIDYVKGQLTELLTRYGTIDLLWFDGEWERTAEEWHAAELRELIKSLQPEVIINDRLPGRGDYATPEQGMPRRPPGGPWELCLTMSDSWAYRPDDLDYKSPRLLAGYLSEVVSRGGNFLLNIGPRGDGSLVPTEVATLKRLGAWLASHGESLFGAGPASERLDFHGPATQHDDTIYLHLLLQPQDLLVVRNVPIGSVAGVRMLGTEQPLEYSVNEEVHKANGAEDEPLGELFITVPPPTGALIDVIAIELSNSRKDESRAGLV

SEQ ID NO: 6 - protein, 429 aa, Streptomyces scabiei
TMQPWFPAAKLGIFIHYGIYAVDGVPESWSFYWGEVTHERYMKQLDGFTASAYDPSAWAGLFARAGAGYAVLTARHHDGVALWDTAHGELDVVRRTPAGRDLIGGYVDALRERGLKVGLYYSHSDWNHPDYASLRHPEIDRWSHTHPGGNDANPYAHAAPGEEDPAAWERYLAYRDGQVKELVERFRPDLLWFDGEWERTEQQWRITELADLILAENPDTVLNARMLGRGDYATPEQGVPLEAPGGPWELCLTINDSWGFQHADHNHKSVRQLVRYFTETIGMGGNLLLDVGPRADGTIPAEQVERLEGLGAWIARHSEAVHGTVAGLPAGHHYGPSTLSADRRTLYLTCFDVPRESVAVRGLRNPVRRVTVVGTGTELGHRVIGGLDAVPGVTWIDAPEEADLDPYATVLALELEGELDLYRGTGRDV

SEQ ID NO: 7 - protein, 464 aa, Opitutus terrae
AVTPRSLLTSLSWLRLTGALVAATILGIVVRAAEPSTPMITDRPDMTWFTDARLGIFIHWGIYSEGKGSESWAFHDGQMPYDEYMAQAKTFTAAKYDPAHWAELFKAAGARYTVLTSKHHDGFALWDTKQSALNAKDGAPAGRDLIGPYCEAMRAQGLKVGLYFSHLDWSHPDYASVFNAAGANDPAGRKNRFSYPQGPENPAAWERFLAFHRAQLREITERFHPDLLWFDGDWERSAEQWRMKELRAQLKSWLPDVILNSRMQGYGDYATPEQALPVRPPTGPWELCMTINDSWGYQAKDRNFKTVRQIVRLLTECASQGGNLLLDVGPRSDGTITPEQEQILRALGRWTSKHAGALYGTTAGIPKDYYYGPSLLNKARDVLYLVCYDRPVDGLYVKGIRNPVKRASVEGGGELTHRRFMKAEWAHQPGIVIVDLPEEAADPDATIVRLELDGPIELLEPGTV

SEQ ID NO: 8 - protein, 24 aa, synthetic construct
MSYYHHHHHHLESTSLYKKAGSGS

SEQ ID NO: 9 - protein, 18 aa, synthetic construct
MSYYLESTSLYKKAGSGS

SEQ ID NO: 10 - protein, 352 aa, synthetic construct
MQTQPLKSVFSIDAGRKYFSVEQLEELVAKASQNGYTDVQLILGNDGLRFILDDMSVNVNGKKYNHNRVSKAIQRGNNAYYNDPNGNALTQKEMDRLLAFAKARNINIIPVINSPGHMDALLVAMEKLAIKNPAFDGSKRPVDLGNQKAVNFTKAIISKYVAYFSAHSEIFNFGGDEYANDVDTGGWAKLQSSGRYKDFVAYANDLAKIIKDAGMQPMSFNDGIYYNSDDSFGTFDPEIIISYWTAGWSGYDVAKPEYFVQKGHKIFNTNDAWYWVAGNVDSGIYQYDDALANMSKKAFTDVPAGSPNLPIIGSIQCVWYDDPRRDYDFERIYTLMDTFSENYREYMVVKNH

SEQ ID NO: 11 - protein, 837 aa, synthetic construct
MNGVQKGMVFKVGNNLSTRKGENRETIVSWLGLSLLVGLAFILFSLFHQPMISQANEPTQEKHFMVYYRAWRDKTMQGVNTTLPDENWLTMHDIPYGIDIVNVFSYVPKGQEALAQPFYDTLKNEYAPALHARGVRLVRGIDYSELLKVPYAGTTPTEAEFDAYAKELLTKFVDDLGIDGLDIDMETRPSEKDIVLSNGVIRALSKYIGPKSGTDRPFLYDTNAEYLPPLQDVSDCFDFLAYQQYGSDDQRTQRALNNLSPVLNGERFVPGLTFPEEQDRNRWYDTKEPYMESNMYKVARYSYENNLGGMFLYALDRDGRTYNEDDLNQIKPSNLLWTKTAIAESKGVSLAEMKAAAQHYLKRISYANTDLEAQNKAAEAVTQATTLYDVNKAILGGDYGQGLSNTYDAELEKGLLAIDLTTLYRALDQAVAAIEKAESYTPETIQALQTTKESVATELAGKTYTAAQVTTWQTEVQTALDNLKEKQTQPLKSVFSIDAGRKYFSVEQLEELVAKASQNGYTDVQLILGNDGLRFILDDMSVNVNGKKYNHNRVSKAIQRGNNAYYNDPNGNALTQKEMDRLLAFAKARNINIIPVINSPGHMDALLVAMEKLAIKNPAFDGSKRPVDLGNQKAVNFTKAIISKYVAYFSAHSEIFNFGGDEYANDVDTGGWAKLQSSGRYKDFVAYANDLAKIIKDAGMQPMSFNDGIYYNSDDSFGTFDPEIIISYWTAGWSGYDVAKPEYFVQKGHKIFNTNDAWYWVAGNVDSGIYQYDDALANMSKKAFTDVPAGSPNLPIIGSIQCVWYDDPRRDYDFERIYTLMDTFSENYREYMVVKNH

SEQ ID NO: 12 - protein, 622 aa, synthetic construct
MNGVQKGMVFKVGNNLSTRKGENRETIVSWLGLSLLVGLAFILFSLFHQPMISQANEPTQEKHFMVYYRAWRDKTMQGVNTTLPDENWLTMHDIPYGIDIVNVFSYVPKGQEALAQPFYDTLKNEYAPALHARGVRLVRGIDYSELLKVPYAGTTPTEAEFDAYAKELLTKFVDDLGIDGLDIDMQTRPSEKDIVLSNGVIRALSKYIGPKSGTDRPFLYDTNAEYLPPLQDVSDCFDFLAYQQYGSDDQRTQRALNNLSPVLNGERFVPGLTFPEEQDRNRWYDTKEPYMESNMYKVARYSYENNLGGMFLYALDRDGRTYNEDDLNQIKPSNLLWTKTAIAESKGVSLAEMKAAAQHYLKRISYANTDLEAQNKAAEAVTQATTLYDVNKAILGGDYGQGLSNTYDAELEKGLLAIDLTTLYRALDQAVAAIEKAESYTPETIQALQTTKESVATELAGKTYTAAQVTTWQTEVQTALDNLKEKQTQPLKSVFSIDAGRKYFSVEQLEELVAKASQNGYTDVQLILGNDGLRFILDDMSVNVNGKKYNHNRVSKAIQRGNNAYYNDPNGNALTQKEMDRLLAFAKARNINIIPVINSPGHMDALLVAMEKLAIKNPAFDG

SEQ ID NO: 13 - protein, 995 aa, synthetic construct
MDKHLLVKRTLGCVCAATLMGAALATHHDSLNTVKAEEKTVQVQKGLPSIDSLHYLSENSKKEFKEELSKAGQESQKVKEILAKAQQADKQAQELAKMKIPEKIPMKPLHGSLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

SEQ ID NO: 14 - protein, 995 aa, synthetic construct
MDKHLLVKRTLGCVCAATLMGAALATHHDSLNTVKAEEKTVQVQKGLPSIDSLHYLSENSKKEFKEELSKAGQESQKVKEILAKAQQADKQAQELAKMKIPEKIPMKPLHGSLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVQVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

SEQ ID NO: 15 - protein, 808 aa, synthetic construct
MEEKTVQTGKTDQQVGAKLVQEIREGKRGPLYAGYFRTWHDRASTGIDGKQQHPENTMAEVPKEVDILFVFHDHTASDSPFWSELKDSYVHKLHQQGTALVQTIGVNELNGRTGLSKDYPDTPEGNKALAAAIVKAFVTDRGVDGLDIDIEHEFTNKRTPEEDARALNVFKEIAQLIGKNGSDKSKLLIMDTTLSVENNPIFKGIAEDLDYLLRQYYGSQGGEAEVDTINSDWNQYQNYIDASQFMIGFSFFEESASKGNLWFDVNEYDPNNPEKGKDIEGTRAKKYAEWQPSTGGLKAGIFSYAIDRDGVAHVPSTYKNRTSTNLQRHEVDNISHTDYTVSRKLKTLMTEDKRYDVIDQKDIPDPALREQIIQQVGQYKGDLERYNKTLVLTGDKIQNLKGLEKLSKLQKLELRQLSNVKEITPELLPESMKKDAELVMVGMTGLEKLNLSGLNRQTLDGIDVNSITHLTSFDISHNSLDLSEKSEDRKLLMTLMEQVSNHQKITVKNTAFENQKPKGYYPQTYDTKEGHYDVDNAEHDILTDFVFGTVTKRNTFIGDEEAFAIYKEGAVDGRQYVSKDYTYEAFRKDYKGYKVHLTASNLGETVTSKVTATTDETYLVDVSDGEKVVHHMKLNIGSGAIMMENLAKGAKVIGTSGDFEQAKKIFDGEKSDRFFTWGQTNWIAFDLGEINLAKEWRLFNAETNTEIKTDSSLNVAKGRLQILKDTTIDLEKMDIKNRKEYLSNDENWTDVAQMDDAKAIFNSKLSNVLSRYWRFCVDGGASSYYPQYTELQILGQRLSNDVANTLKD

SEQ ID NO: 16 - protein, 808 aa, synthetic construct
MEEKTVQTGKTDQQVGAKLVQEIREGKRGPLYAGYFRTWHDRASTGIDGKQQHPENTMAEVPKEVDILFVFHDHTASDSPFWSELKDSYVHKLHQQGTALVQTIGVNELNGRTGLSKDYPDTPEGNKALAAAIVKAFVTDRGVDGLDIMIEHEFTNKRTPEEDARALNVFKEIAQLIGKNGSDKSKLLIMDTTLSVENNPIFKGIAEDLDYLLRQYYGSQGGEAEVDTINSDWNQYQNYIDASQFMIGFSFFEESASKGNLWFDVNEYDPNNPEKGKDIEGTRAKKYAEWQPSTGGLKAGIFSYAIDRDGVAHVPSTYKNRTSTNLQRHEVDNISHTDYTVSRKLKTLMTEDKRYDVIDQKDIPDPALREQIIQQVGQYKGDLERYNKTLVLTGDKIQNLKGLEKLSKLQKLELRQLSNVKEITPELLPESMKKDAELVMVGMTGLEKLNLSGLNRQTLDGIDVNSITHLTSFDISHNSLDLSEKSEDRKLLMTLMEQVSNHQKITVKNTAFENQKPKGYYPQTYDTKEGHYDVDNAEHDILTDFVFGTVTKRNTFIGDEEAFAIYKEGAVDGRQYVSKDYTYEAFRKDYKGYKVHLTASNLGETVTSKVTATTDETYLVDVSDGEKVVHHMKLNIGSGAIMMENLAKGAKVIGTSGDFEQAKKIFDGEKSDRFFTWGQTNWIAFDLGEINLAKEWRLFNAETNTEIKTDSSLNVAKGRLQILKDTTIDLEKMDIKNRKEYLSNDENWTDVAQMDDAKAIFNSKLSNVLSRYWRFCVDGGASSYYPQYTELQILGQRLSNDVANTLKD

SEQ ID NO: 17 - protein, 453 aa, synthetic construct
MVIDQKDIPDPALREQIIQQVGQYKGDLERYNKTLVLTGDKIQNLKGLEKLSKLQKLELRQLSNVKEITPELLPESMKKDAELVMVGMTGLEKLNLSGLNRQTLDGIDVNSITHLTSFDISHNSLDLSEKSEDRKLLMTLMEQVSNHQKITVKNTAFENQKPKGYYPQTYDTKEGHYDVDNAEHDILTDFVFGTVTKRNTFIGDEEAFAIYKEGAVDGRQYVSKDYTYEAFRKDYKGYKVHLTASNLGETVTSKVTATTDETYLVDVSDGEKVVHHMKLNIGSGAIMMENLAKGAKVIGTSGDFEQAKKIFDGEKSDRFFTWGQTNWIAFDLGEINLAKEWRLFNAETNTEIKTDSSLNVAKGRLQILKDTTIDLEKMDIKNRKEYLSNDENWTDVAQMDDAKAIFNSKLSNVLSRYWRFCVDGGASSYYPQYTELQILGQRLSNDVANTLKD

SEQ ID NO: 18 - protein, 783 aa, synthetic construct
MQTENTTEQRLNAWFTDAKLGIFIHWGYYGVNGITESWSMYHKRISHTDYLKQGQKFTASNYNPDQWLDLFERVGAKYVVLTTKHHDGVALWNTKYSNLSTVKHASARKDVLTPFVEAVRKKNIKLGLYYSLCDWSHPDYQPVTFQRRDVRKLYPQKGQQKMTPWERFTAFNFNQLEELSQFKPDLWWFDGDWEHKPEEWKSKEIKEKLLFWNPQTVVNSRLNQYGDYKTPEQGVPVVRPEGAWEFCMTMNDNWGYFPTDTNYKPIAQIIRTFVEVIGSGGNLLLNVGPKPDGTIPQEQVLRLEALGNWISKHKTAVFDTQAGLPYGHFFGPTLLSKDKTKIYLCLFDNPKNYILLKGIQNKVKSIKVLGAEQKISFERNGGAAWNNIPGILRISVPADQSLDPYVTVLEVHLEGELSLYRGHGNAVELNMVQTQPLKSVFSIDAGRKYFSVEQLEELVAKASQNGYTDVQLILGNDGLRFILDDMSVNVNGKKYNHNRVSKAIQRGNNAYYNDPNGNALTQKEMDRLLAFAKARNINIIPVINSPGHMDALLVAMEKLAIKNPAFDGSKRPVDLGNQKAVNFTKAIISKYVAYFSAHSEIFNFGGDEYANDVDTGGWAKLQSSGRYKDFVAYANDLAKIIKDAGMQPMSFNDGIYYNSDDSFGTFDPEIIISYWTAGWSGYDVAKPEYFVQKGHKIFNTNDAWYWVAGNVDSGIYQYDDALANMSKKAFTDVPAGSPNLPIIGSIQCVWYDDPRRDYDFERIYTLMDTFSENYREYMVVKNH

SEQ ID NO: 19 - protein, 899 aa, synthetic construct
MQTENTTEQRLNAWFTDAKLGIFIHWGYYGVNGITESWSMYHKRISHTDYLKQGQKFTASNYNPDQWLDLFERVGAKYVVLTTKHHDGVALWNTKYSNLSTVKHASARKDVLTPFVEAVRKKNIKLGLYYSLCDWSHPDYQPVTFQRRDVRKLYPQKGQQKMTPWERFTAFNFNQLEELSQFKPDLWWFDGDWEHKPEEWKSKEIKEKLLFWNPQTVVNSRLNQYGDYKTPEQGVPVVRPEGAWEFCMTMNDNWGYFPTDTNYKPIAQIIRTFVEVIGSGGNLLLNVGPKPDGTIPQEQVLRLEALGNWISKHKTAVFDTQAGLPYGHFFGPTLLSKDKTKIYLCLFDNPKNYILLKGIQNKVKSIKVLGAEQKISFERNGGAAWNNIPGILRISVPADQSLDPYVTVLEVHLEGELSLYRGHGNAVELNMVLEAQNKAAEAVTQATTLYDVNKAILGGDYGQGLSNTYDAELEKGLLAIDLTTLYRALDQAVAAIEKAESYTPETIQALQTTKESVATELAGKTYTAAQVTTWQTEVQTALDNLKEKQTQPLKSVFSIDAGRKYFSVEQLEELVAKASQNGYTDVQLILGNDGLRFILDDMSVNVNGKKYNHNRVSKAIQRGNNAYYNDPNGNALTQKEMDRLLAFAKARNINIIPVINSPGHMDALLVAMEKLAIKNPAFDGSKRPVDLGNQKAVNFTKAIISKYVAYFSAHSEIFNFGGDEYANDVDTGGWAKLQSSGRYKDFVAYANDLAKIIKDAGMQPMSFNDGIYYNSDDSFGTFDPEIIISYWTAGWSGYDVAKPEYFVQKGHKIFNTNDAWYWVAGNVDSGIYQYDDALANMSKKAFTDVPAGSPNLPIIGSIQCVWYDDPRRDYDFERIYTLMDTFSENYREYMVVKNH

SEQ ID NO: 20 - protein, 995 aa, synthetic construct
MQTENTTEQRLNAWFTDAKLGIFIHWGYYGVNGITESWSMYHKRISHTDYLKQGQKFTASNYNPDQWLDLFERVGAKYVVLTTKHHDGVALWNTKYSNLSTVKHASARKDVLTPFVEAVRKKNIKLGLYYSLCDWSHPDYQPVTFQRRDVRKLYPQKGQQKMTPWERFTAFNFNQLEELSQFKPDLWWFDGDWEHKPEEWKSKEIKEKLLFWNPQTVVNSRLNQYGDYKTPEQGVPVVRPEGAWEFCMTMNDNWGYFPTDTNYKPIAQIIRTFVEVIGSGGNLLLNVGPKPDGTIPQEQVLRLEALGNWISKHKTAVFDTQAGLPYGHFFGPTLLSKDKTKIYLCLFDNPKNYILLKGIQNKVKSIKVLGAEQKISFERNGGAAWNNIPGILRISVPADQSLDPYVTVLEVHLEGELSLYRGHGNAVELNMVDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

SEQ ID NO: 21 - protein, 884 aa, synthetic construct
MQTENTTEQRLNAWFTDAKLGIFIHWGYYGVNGITESWSMYHKRISHTDYLKQGQKFTASNYNPDQWLDLFERVGAKYVVLTTKHHDGVALWNTKYSNLSTVKHASARKDVLTPFVEAVRKKNIKLGLYYSLCDWSHPDYQPVTFQRRDVRKLYPQKGQQKMTPWERFTAFNFNQLEELSQFKPDLWWFDGDWEHKPEEWKSKEIKEKLLFWNPQTVVNSRLNQYGDYKTPEQGVPVVRPEGAWEFCMTMNDNWGYFPTDTNYKPIAQIIRTFVEVIGSGGNLLLNVGPKPDGTIPQEQVLRLEALGNWISKHKTAVFDTQAGLPYGHFFGPTLLSKDKTKIYLCLFDNPKNYILLKGIQNKVKSIKVLGAEQKISFERNGGAAWNNIPGILRISVPADQSLDPYVTVLEVHLEGELSLYRGHGNAVELNMVVIDQKDIPDPALREQIIQQVGQYKGDLERYNKTLVLTGDKIQNLKGLEKLSKLQKLELRQLSNVKEITPELLPESMKKDAELVMVGMTGLEKLNLSGLNRQTLDGIDVNSITHLTSFDISHNSLDLSEKSEDRKLLMTLMEQVSNHQKITVKNTAFENQKPKGYYPQTYDTKEGHYDVDNAEHDILTDFVFGTVTKRNTFIGDEEAFAIYKEGAVDGRQYVSKDYTYEAFRKDYKGYKVHLTASNLGETVTSKVTATTDETYLVDVSDGEKVVHHMKLNIGSGAIMMENLAKGAKVIGTSGDFEQAKKIFDGEKSDRFFTWGQTNWIAFDLGEINLAKEWRLFNAETNTEIKTDSSLNVAKGRLQILKDTTIDLEKMDIKNRKEYLSNDENWTDVAQMDDAKAIFNSKLSNVLSRYWRFCVDGGASSYYPQYTELQILGQRLSNDVANTLKD

(19) United States
(12) Patent Application Publication
(10) Pub. No.: US 2026/0297642 A1
(43) Pub. Date: Oct. 1, 2026

(54) Fucosidase Enzymes For Therapeutic Applications

(71) Applicants: VIB VZW, Gent (BE); Universiteit Gent, Gent (BE)

(72) Inventors: Nico Callewaert, Gent (BE); Berre Van Moer, Gent (BE); Wim Nerinckx, Gent (BE); Hendrik Grootaert, Gent (BE); Jasper Zuallaert, Gent (BE)

(21) Appl. No.: 19/163,062

(22) PCT Filed: Mar. 8, 2024
(86) PCT No.: PCT/EP2024/056177

§ 371 Date: Sep. 8, 2025

(30) Foreign Application Priority Data
Mar. 8, 2023 (EP) 23160633.6

(51) Int. Cl. C12P 21/00; C07K 16/00; C12N 9/24; C12N 11/00

(52) U.S. Cl. CPC C12P 21/005; C07K 16/00; C12N 9/2402; C12N 11/00; C07K 2317/14; C07K 2317/41; C07K 2317/569; C12Y 302/01051; C12Y 302/01052

(57) ABSTRACT

The present invention relates to the field of glyco-engineering of antibodies, more specifically the field of fucosidases capable of removing core fucose from N-glycan substrates present on antibody Fc regions. The present invention in particular relates to a composition for defucosylation of an N-glycosylated Fc-containing protein, said composition comprising the Capnocytophaga canimorsus Glycosyl Hydrolase family 29 (GH29) α-fucosidase or a variant thereof, and a pretreatment enzyme. More specifically, said composition comprises a pretreatment enzyme for trimming the N-glycosylated Fc-containing protein sample to trimannosyl-glycan substrates on said Fc-containing proteins, for subsequent defucosylation by the Capnocytophaga canimorsus GH29 enzyme. The invention further specifies methods and uses of said enzymes and compositions for production of defucosylated Fc-containing proteins and their therapeutic utility.

### CROSS REFERENCE TO RELATED APPLICATIONS

This application is a national phase entry under 35 U.S.C. § 371 of International Patent Application PCT/EP2024/056177, filed Mar. 8, 2024, designating the United States of America and published in English as International Patent Publication WO2024/184512 on Sep. 12, 2024, which claims the benefit under Article 8 of the Patent Cooperation Treaty to European Patent Application Serial No. 23160633.6, filed Mar. 8, 2023, the entireties of which are hereby incorporated by reference.

### INCORPORATION BY REFERENCE

The ST.26 XML Sequence listing named “10488-11056-US-Sequence Listing ST26”, created on Mar. 7, 2024, and having a size of 32,768 bytes, is hereby incorporated herein by this reference in its entirety.

### FIELD OF THE INVENTION

The present invention relates to the field of glyco-engineering of antibodies, more specifically the field of fucosidases capable of removing core fucose from N-glycan substrates present on antibody Fc regions. The present invention in particular relates to a composition for defucosylation of an N-glycosylated Fc-containing protein, said composition comprising the Capnocytophaga canimorsus Glycosyl Hydrolase family 29 (GH29) α-fucosidase or a variant thereof, and a pretreatment enzyme. More specifically, said composition comprises a pretreatment enzyme for trimming the N-glycosylated Fc-containing protein sample to trimannosyl-glycan substrates on said Fc-containing proteins, for subsequent defucosylation by the Capnocytophaga canimorsus GH29 enzyme. The invention further specifies methods and uses of said enzymes and compositions for production of defucosylated Fc-containing proteins and their therapeutic utility.

### BACKGROUND

Antibodies binding to their target antigen elicit effector functions, performed by immune cells. One of said effector functions concerns antibody dependent cellular cytotoxicity (ADCC), wherein natural killer cells induce cell death in aberrant cells, which are marked by opsonization with antibodies binding to a cell surface antigen (Johnson, & Glennie. 2003. Semin. Oncol. 30(1 Suppl 2):3-8). Immune cells specifically recruit to those sites through their Fcgamma receptors (FcγRs) which bind to the Fc domain of antibodies, specifically FcγRIIIa (CD16a) on natural killer cells (Sulica, et al. 2001. Int Rev Immunol. 2001 June; 20(3-4):371-414.). Antibody therapies are suboptimal as anti-tumor treatments when their N297 N-glycan is core-fucosylated, since the a1-6 linked saccharide drastically reduces the affinity between antibodies and FcgRIIIa, due to steric hindrance to the N162 N-glycan of the receptor (Shields, et al. 2002; J. Biol. Chem. 277, 26733-26740; Shinkawa, et al. 2003; J. Biol. Chem. 278, 3466-3473). The absence of core fucose on the Fc N-glycan has been shown to increase IgG1 Fc binding affinity to the FcγRIIIa present on immune effector cells such as natural killer cells and lead to enhanced ADCC activity. As such, various strategies have focused on producing afucosylated antibodies to improve therapeutic efficacy, for applications in cancer treatment, but also in inherited immunodeficiency diseases.

Generating non-fucosylated recombinant antibodies therefore has been achieved by employing host cells during production which are incapable of incorporating core-fucose, or which are devoid in the metabolic pathways to provide the required sugar donor GDP-fucose. CHO cells, the main production hosts for commercial antibodies, commonly incorporate core-fucose in IgG (Mori et al. 2007; Cytotechnology 55(2-3):109-14), making clinically relevant monoclonal antibodies expressed in CHO less efficient as cancer treatments. In order to reduce the level of fucosylation in CHO cells, manipulations during protein productions have been carried out. The most straightforward, genetic solution to remove fucosylation is implemented in the POTELLIGENT technology in which the responsible fucosyltransferase gene (FUT8) is knocked out in CHO cells (Yamane-Ohnuki, et al., 2004; Biotechnology and Bioengin. 87(5) 614-622). As FUT8 is only responsible for core a1-6 fucosylation, other fucosylation products such as the Lewis antigens are preserved. Alternative approaches for obtaining defucosylated antibodies in hosts involve changes in the fucose metabolism (e.g. blocking de novo synthesis through knocking out or mutating GDP-L-fucose synthase and/or GDP-mannose 4,6 dehydratase), mutation of the N-glycosylation pathway (e.g. removing GnT-I required for GlcNac required for core-fucosylation), applying fucosylation inhibitors during manufacturing (e.g. 2F-peracetyl-fucose), addition of small molecules to control relevant enzyme expression during production, or increasing the level of bisection on antibodies, as implemented in GlycoMAb's technology. Next to engineering cells or production processes, non-fucosylated antibodies can also be generated by using host cells that intrinsically have a reduced fucosylation activity (e.g. CHO Lec13 cells which have a deficient GMDS activity). Additionally, most non-mammalian expression systems differ in fucosylation capacity. For instance, yeast glycoproteins are not incorporated with fucose, but only consist of GlcNAc and terminally positioned mannose residues. Next to in cellulo approaches, enzymatic alternatives have been disclosed as well, which can be implemented after antibody production. Yet, none have been anticipated that hydrolyze the core-fucose from antibodies carrying intact biantennary N-glycans.

Enzymes capable of hydrolyzing fucose residues from N-glycans belong to the glycosyl hydrolase (GH) families GH29 and GH95 (Grootaert, et al. 2020; Glycobiology, 30, 9, 735-745; Lombard, et al. 2014; Nucleic Acids Res. 42, D490-D495). While the GH95 family mainly houses a1-2 fucosidases, the GH29 family contains fucosidases with a wider substrate specificity: α1-2/3/4/6 (McCarter and Withers, 1994; Curr. Opin. Struct. Biol., 4, 885-892). As core-fucosylation is incorporated in an α1-6 glycosidic linkage to the innermost GlcNAc residue, all research to find enzymes active on fucosylated IgG carrying biantennary N-glycans has started from GH29 family members.

A first group of enzymes (bovine kidney fucosidase, L. casei fucosidase AlfC and B. fragilis fucosidase BfFucH) removes core-fucose from IgG, however only after using endoglucosaminidases (ENGases) such as Endo S to trim the N-glycan into a fucosylated GlcNAc residue (Huang et al., 2012; Am. Chem. Soc. 134, 29, 12308-12318; Li et al., 2016; J Biol Chem. 291(32): 16508-16518; Tsai et al., 2017; ACS Chem. Biol. 2017, 12, 1, 63-72). Upon ENGase trimming, the structure of the Fc domain is however similarly collapsed as non-glycosylated IgG, thereby reducing effector functionality (Allhorn et al., 2008; PLOS One.; 3(1): e1413; Sjögren, et al., 2015; Glycobiology, 25, Issue 10, 1053-1063). Restoring the structure to the level of naturally N-glycosylated antibodies can be done through transglycosylation of the GlcNAc stump with chemically activated sugar donors. However, this final step would make the overall strategy very expensive.

The NEB fucosidase O enzyme from the Omnitrophica bacterium OLB16 is the only GH29 enzyme that indicated to be active in (partially) removing core-fucose on N-glycosylated IgG, with its optimal activity window at pH 5.5 and at 50° C. Complete fucose removal was demonstrated for RFMS-labeled forms of hIgGs, though not for non-trimmed or non-labelled hIgGs (Vainauskas et al. 2018; Sci Rep. 8(1):9504; Taron et al. U.S. Pat. No. 10,260,056B2).

Ideally, enzyme activity for removing fucosidase is achieved on the GlcNAC₂Man₃GlcNAC₂Fuc substrate being the N-glycan in the main product from CHO based manufacturing, which is still problematic. So there is a need to further shed light on the GH29 fucosidase substrate specificity as to address the right enzymes in efficiently, and at large-scale allow defucosylation of therapeutic antibodies for anti-tumor treatments.

### SUMMARY OF THE INVENTION

In this study, the screening of several enzymes from the Glycosyl Hydrolase 29 (GH29) family of fucosidases selected based on AlphaFold2 models for their activity on VHH-Fc-Man₃GlcNAC₂Fuc antibodies, revealed Capnocytophaga canimorsus GH29 α-fucosidase (AEK23747; SEQ ID NO:1 (lacking signal peptide), also referred to as ‘H2’ or ‘CC-H2’ herein) to have optimal activity on this N-glycan antibody substrate under physiological conditions, i.e. at pH 7 and 37° C., whereas several other enzymes from the GH29 family, specifically phylogenetically closely related to C. canimorsus, did not show any activity at either physiological or acidic pH. So far, only fucosidase O (commercially available from NEB) from the α-L-fucosidase GH29 family has been reported to have activity on human IgG substrates with N-glycans partially or not trimmed to GlcNac, under certain conditions.

The invention relates to the GH29 CC-H2 fucosidase capability to act on N-glycosylated Fc-containing proteins, in particular antibodies, thus N-glycosylated antibody substrates, which was remarkable as none of many further enzymes showed specificity for these N-glycan substrates. Since this CC-H2 enzyme is positioned in a totally different branch of the GH29 family phylogenetic tree as compared to Fucosidase O, its substrate specificity could thus not be predicted based on the activity observed for the phylogenetically distant Fuc O. Moreover, the newly identified enzyme with activity on N-glycans of Fc-containing protein has a pH optimum close to the physiological range whereas Fucosidase O operates optimally at acidic pH (4.5-5.5).

The C. canimorsus Fuc H2 enzyme activity described herein was further optimized in view of increasing its efficiency towards defucosylation of N-glycosylated Fc-containing proteins, partially trimmed, to provide for the first fucosidase composition with optimal productivity at physiological conditions.

The invention herein describes a novel solution for defucosylation of antibodies carrying the larger GlcNAC₂Man₃GlcNAc₂Fuc N-glycan, as this is the main glycoform incorporated on CHO-produced antibodies, by providing a composition combining the Capnocytophaga canimorsus (‘H2’) α-fucosidase with a pretreating enzyme, preferably a hexosaminidase, for pre-trimming the GlcNAC₂Man₃GlcNAC₂Fuc, as to provide Man₃GlcNAc₂ Fuc N-glycan substrate for CC-H2 fucosidase. Alternatively, said pretreatment enzyme of the composition is a scavenger enzyme, as described herein, or the pretreatment enzyme(s) comprise both, hexosaminidase and scavenging activity. So by combining multiple enzyme activities, or by fusion of said enzymes or enzyme domains, complete defucosylation of intact N-glycosylated antibodies as present in mammalian and human cells is achieved by utilizing a single enzyme composition.

### DESCRIPTION OF THE FIGURES

The patent or application file contains at least one drawing executed in color. Copies of this patent or patent application publication with color drawing(s) will be provided by the Office upon request and payment of the necessary fee.

The drawings described are only schematic and are non-limiting. In the drawings, the size of some of the elements may be exaggerated and not drawn on scale for illustrative purposes.

FIGS. 1A-1F. PyMol snapshots of fucosidases in the GH29 collection, with a docked Man₃GlcNAc₂Fuc N-glycan. Fucosidases are coloured according to the prediction confidence: AlfC (FIG. 1A), Fucosidase O (FIG. 1B), H2 (FIG. 1C), A8 (FIG. 1D), A2 (FIG. 1E) and C9 (FIG. 1F). The N-glycan is shown as sticks in each panel. It is colored based on the specific monosaccharide residue in the N-glycan: fucose black, GlcNAc blue and mannose green.

FIG. 2. Jack Bean hexosaminidase digest of intact VHH-Fcs removed most terminal GlcNAc residues. Overnight Jack Bean hexosaminidase (Agilent) or mock digest of intact ExpiCHO-produced VHH-Fc, aiming to remove GlcNAc residues from GlcNAc2Man₃GlcNAc₂ Fuc glycans to generate Man₃GlcNAC₂Fuc. After the digest, N-glycans were released by PNGaseF treatment and detected via capillary electrophoresis.

FIGS. 3A-3C. Identification of three fucosidases active on VHH-Fc N-glycan, labeled with APTS. Prior to APTS-labeling, intact VHH-Fc was treated with hexosaminidase from Jack Bean. This is indicated as (VHH-Fc+Hex) next to the panels. Next, fucosidases selected from our GH29 collection were incubated overnight with the APTS-labeled N-glycans and subsequently ran on CE.

FIG. 4. Fucosidase H2 and Fuc O partially remove core-fucose from intact VHH-Fc. VHH-Fc, pretreated with hexosaminidase (Jack's Bean), was incubated overnight at 37 C with fucosidases (H2, fuc O and A8) in 5 mM CaCl2) and 50 mM NaAcO⁻ at pH 5.5.

FIGS. 5A-5C. Phylogenetic tree of the in house GH29 family collection. The collection was pruned to include the maximal diversity with the fewest sequences. Additionally, sequences for fucosidase O and five fucosidases from GH95 (‘Extra’ enzymes indicated in brown in FIG. 5C) were added. The fucosidases included in the screening for defucosylation of VHH-Fc (Endo E GH20 pretreated) are indicated in bold. Fucosidase O and AlfC from L. casei BL23 are indicated in green in FIG. 5B, while H2 (Capnocytophaga canimorsus) is indicated in blue in FIG. 5A. Phylogeny constructed with MEGA11 software.

FIG. 6. Recombinantly produced Endo E GH20 domain generates VHH-Fc with Man₃GlcNAc₂Fuc. VHH-Fc was treated with Endo E GH20 in PBS for 24 h at 37 C. Next, the VHH-Fc was purified by protein A chromatography and subjected to DSA FACE for glycan analysis.

FIG. 7. Activity screening for fucosidases selected from the GH29 family collection. Fucosidases were incubated for 30 minutes with 1 mM chloro-nitrophenol-fucose (CNP-fucose) in 50 mM sodium phosphate pH 7. Units were determined based on comparison with a dilution series of H2 #1.

FIG. 8. Fucosidases H2 and O are capable of removing core-fucose from VHH-Fc, in a pH dependent manner. VHH-Fc was pretreated with Endo E GH20 and purified by protein A chromatography. Next the VHH-Fc was incubated overnight at 37° C. with the produced fucosidases in Na₂HPO₄ PH 7 or MES pH 5. Next, N-glycans were extracted, labeled and analyzed by capillary electrophoresis (CE) and the area for the non-fucosylated Man₃GlcNAc₂ peak plotted for each condition. For simplicity, only the profiles for close relatives of fucosidase H2, H2 and Fuc O are shown. Bars from left to right, in each pH condition, represent the intensity of Man₃GlcNac₂ defucosylated products for samples containing the following enzymes: ‘none’, H2, FucO, A4, A9, A11, A12, H3.

FIG. 9. Fucosidase H2 is most active on VHH-Fc carrying Man₃GlcNAc₂Fuc at physiological pH. The activity of fucosidase H2 was compared in a range of pH conditions in different buffers. The substrate of interest was VHH-Fc pretreated with Endo E GH20 to carry some level of Man₃GlcNAc₂Fuc. Fucosidase H2 was incubated overnight at 37 C in one of the following buffer: Mcllvaine buffer (mix of 0.1M citric acid and 0.2M Na₂HPO₄), 40 mM NH₄AcO⁻, 50 mM Na₂HPO₄ (NaPi), 50 mM Tris. Next, the N-glycans were analyzed by DSA FACE and the area for the non-fucosylated Man₃GlcNAc₂ peak plotted for each condition.

FIG. 10. Fucosidase H2 activity on VHH-Fc carrying Man GlcNAc₂Fuc is reduced in the presence of EDTA, but not in citrate, possibly due to activity improvement by nickel or calcium ions. VHH-Fc pretreated with Endo E GH20, was incubated overnight at 37 C with fucosidase H2 at pH 7 in the presence of varying amounts of EDTA or citrate, or 10 mM of bivalent metals as SO₄²⁻ ions. The control condition with no additives is shown left with a black bar. Next, the N-glycans were analyzed by DSA FACE and the area for the non-fucosylated Man₃GlcNAc₂ peak plotted for each condition.

FIG. 11. Defucosylation of VHH-Fc, carrying Man₃GlcNAc₂Fuc is not influenced by urea, GlcNAc or mannose residues, but is reduced in DMSO. VHH-Fc pretreated with Endo E GH20, was incubated overnight at 37 C with fucosidase H2 at pH 7 in the presence of varying amounts of urea, DMSO, GlcNAc or mannose. The control condition with no additives is shown left with a black bar. Next, the N-glycans were analyzed by DSA FACE and the area for the non-fucosylated Man₃GlcNAc₂ peak plotted for each condition.

FIG. 12. Defucosylation of VHH-Fc, carrying Man₃GlcNAC₂Fuc, can be boosted by repeatedly adding the same amount of fucosidase H2. Two batches H2 #1 and H2 #2 were tested for their defucosylation activity on VHH-Fc pretreated with Endo E GH20, incubated at 37° C. with fucosidase H2 at pH 7. The number of enzyme additions varied between the samples. After the final incubation, the N-glycans were analyzed by DSA FACE and the area for the non-fucosylated Man₃GlcNAc₂ peak plotted for each condition. The second batch of fucosidase H2 is slightly less active than the first batch with VHH-Fc as substrate.

### DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein. The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases ‘in one embodiment’ or ‘in an embodiment’ in various places throughout this specification are not necessarily all referring to the same embodiment but may.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for definitions and terms of the art. Practitioners are particularly directed to Lauc and Wuhrer, High-Throughput Glycomics and Glycoproteomics, Springer Science+Business Media, LLC, part of Springer Nature, Humana New York, NY (2017), for definitions and terms used in glycobiology and glycoproteomic approaches. Unless defined otherwise, all technical and scientific terms used herein have the same meaning as commonly understood by one of ordinary skill in the art (e.g. in molecular biology, biochemistry, structural biology, and/or computational biology).

The terms “protein”, “polypeptide”, and “peptide” are interchangeably used further herein to refer to a polymer of amino acid residues and to variants and synthetic analogues of the same. A monomeric or protomer is defined as a single polypeptide chain from amino-terminal end (also referred to herein as N-term or N-terminus or N-terminal end) to carboxy-terminal end (also referred to herein as C-term or C-terminus or C-terminal end). A “protein subunit” as used herein refers to a monomer or protomer, which may form part of a multimeric protein complex or assembly.

The terms “chimeric polypeptide”, “chimeric protein”, “chimer”, “fusion polypeptide”, “fusion protein”, are used interchangeably herein and refer to a protein that comprises at least two separate and distinct polypeptide components that may or preferably may not originate from the same protein. The term also refers to a non-naturally occurring molecule which means that it is man-made. The term “fused to”, and other grammatical equivalents, such as “covalently linked”, “connected”, “attached”, “ligated”, “conjugated”, and as specifically used herein ‘inserted in’ when referring to a chimeric or fusion polypeptide (as defined herein) refers to any chemical or recombinant mechanism for linking two or more polypeptide components. The fusion of the two or more polypeptide components may be a direct fusion of the sequences or it may be an indirect fusion, e.g. with intervening amino acid sequences or linker sequences, or chemical linkers. The fusion of amino acid residues or (poly) peptides to an Ena protein or insertion into an Ena protein sequence, or to another protein of interest as described herein, may be a covalent peptide bond, or also refer to a fusion obtained by chemical linking. The term “fused to”, as used herein, and interchangeably used herein as “connected to”, “conjugated to”, “ligated to” refers, in particular, to “genetic fusion”, e.g., by recombinant DNA technology, as well as to “chemical and/or enzymatic conjugation” resulting in a stable covalent link.

By “recombinant polypeptide” is meant a polypeptide made using recombinant techniques, i.e., through the expression of a recombinant or synthetic polynucleotide, which may be obtained in vitro and/or in a cellular context. When the chimeric polypeptide or fusion polypeptide or biologically active (i.e. functional) portion thereof is recombinantly produced, it is also preferably enriched, purified or substantially free of culture medium, i.e., the impurities represent less than about 20%, more preferably less than about 10%, and most preferably less than about 5% of the volume of the protein preparation. By “isolated” or “purified” is meant material that is substantially or essentially free from components that normally accompany it in its native state.

“Homologue”, “Homologues” of a protein encompass peptides, oligopeptides, polypeptides, proteins and enzymes having amino acid substitutions, deletions and/or insertions relative to the unmodified or wild-type protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met, also indicated in one-letter code herein) occurs in both sequences to yield the number of matched positions, dividing the number of matched positions by the total number of positions in the window of comparison (i.e., the window size), and multiplying the result by 100 to yield the percentage of sequence identity. A “substitution”, or “mutation” as used herein, results from the replacement of one or more amino acids or nucleotides by different amino acids or nucleotides, respectively as compared to an amino acid sequence or nucleotide sequence of a parental protein or a fragment thereof. It is understood that a protein or a fragment thereof may have conservative amino acid substitutions which have substantially no effect on the protein's activity. The percentage of amino acid identity as provided herein is preferably in view of a window of comparison corresponding to the total length of the native or natural wild-type protein, or of the specific amino acid sequence referred to.

The term “wild-type” refers to a gene or gene product isolated from a naturally occurring source, or included in a cell, cell line or organism. A wild-type gene or gene product is that which is most frequently observed in a population and is thus arbitrarily designed the “normal” or “wild-type” form of the gene or gene product a observed in nature. In contrast, the term “modified”, “engineered”, “mutant” or “variant” refers to a gene or gene product that displays modifications in sequence, post-translational modifications and/or functional properties (i.e., altered characteristics) when compared to the wild-type or naturally-occurring gene or gene product. A knock-out refers to a modified or mutant or deleted gene as to provide for non-functional gene product and/or function. It is noted that naturally occurring mutants or variants may be isolated; these are identified by the fact that they have altered characteristics when compared to the wild-type gene or gene product, and a different sequence as compared to the reference gene or protein.

The term “antibody” refers to an immunoglobulin (Ig) molecule or a molecule comprising an immunoglobulin (Ig) domain, which specifically binds with an antigen. “Antibodies” can further be intact immunoglobulins derived from natural sources or from recombinant sources and can be immunoreactive portions of intact immunoglobulins.

Among the five isotypes of naturally occurring immunoglobulins (Igs), which are IgG, IgA, IgM, IgD, and IgE, IgG comprises the majority, representing 60% of total serum Igs in humans. The human IgG molecule is composed of two identical fragment antigen binding (Fab) domains and one fragment crystallizable (Fc) domain that make it multivalent and multifunctional. The two Fab fragments each consist of a heterodimer of a light chain and the N-terminal part of the heavy chain, whereas the C-terminal half of the two heavy chains dimerizes to form the Fc fragment of the IgG antibody. The N-terminal domains of the Fab fragment are the variable domains (V_{L} and V_{H}) that are responsible for antigen recognition, whereas the C-terminal part of the heavy chains compose the Fc fragment that is responsible for humoral and cellular effector functions. The two Fabs and the Fc are connected by the hinge region, which facilitates the spatial alignment of the three moieties for binding to antigens and effector ligands. “Fc domains” or “Fc-regions” or “Fc-tails”, as interchangeably used herein, and refer to the single Fc chain and/or the dimeric Fc domain of an Fc-containing protein. Specifically in antibodies, said Fc domain is thus responsible for antibody function, and antibody Fc engineering stands for engineering functions of antibodies, which are effector functions, such as antibody-dependent cellular cytotoxicity (ADCC) and antibody-dependent cellular phagocytosis (ADCP), and controlling serum half-life. Engineered Fc domains may therefore be present in the form of mutants or variants containing amino acid substitutions, insertions or deletions as to allow different modifications of the Fc in post-translational modifications, dimerization behavior, effector function, serum half life, among others. To indicate the variations present in Fc domains based on the sequence of naturally occurring IgGs, conventional antibody numbering annotations are known in the art, such as for instance IMGT numbering (LeFranc, 2014; Frontiers in Immunology. 5(22): 1-22), Kabat numbering (Kabat, E. A. et al., Sequences of proteins of immunological interest. 5th Edition-US Department of Health and Human Services, NIH publication n° 91-3242, pp 662, 680, 689(1991)), or preferably used herein EU numbering (Edelman et al. (1969). The covalent structure of an entire gammaG immunoglobulin molecule. Proc Natl Acad Sci USA.; 63:78-85).

The term “active antibody fragment” refers to a portion of any antibody or antibody-like structure that by itself has high affinity for an antigenic determinant, or epitope, and contains one or more complementarity determining regions (CDRs) accounting for such specificity, typically at least 3 CDRs, or in conventional antibodies, defined by 6 CDRs. Non-limiting examples of active antibody fragments include immunoglobulin domains, Fab, F (ab)′2, scFv, heavy-light chain dimers, immunoglobulin single variable domains (ISVDs), Nanobodies (or VHH antibodies), domain antibodies, and single chain structures, such as a complete light chain or complete heavy chain. The term “antibody fragment” and “active antibody fragment” or “functional variant” as used herein refers to a protein comprising an immunoglobulin domain or an antigen-binding domain capable of specifically binding the antigen. Antibodies are typically tetramers of immunoglobulin molecules. The term “immunoglobulin (Ig) domain”, or more specifically “immunoglobulin variable domain” (abbreviated as “IVD”) means an immunoglobulin domain essentially consisting of four “framework regions” which are referred to in the art and herein below as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and herein below as “complementarity determining region 1” or “CDR1”; as “complementarity determining region 2” or “CDR2”; and as “complementarity determining region 3” or “CDR3”, respectively. Thus, the general structure or sequence of an immunoglobulin variable domain can be indicated as follows: FR1-CDR1-FR2-CDR2-FR3-CDR3-FR4. It is the immunoglobulin variable domain(s) (IVDs) that confer specificity to an antibody for the antigen by carrying the antigen-binding site. Typically, in conventional immunoglobulins, a heavy chain variable domain (VH) and a light chain variable domain (VL) interact to form an antigen binding site. In this case, the complementarity determining regions (CDRs) of both VH and VL will contribute to the antigen binding site, i.e. a total of 6 CDRs will be involved in antigen binding site formation. In view of the above definition, the antigen-binding domain of a conventional 4-chain antibody (such as an IgG, IgM, IgA, IgD or IgE molecule; known in the art) or of a Fab fragment, a F(ab′)2 fragment, an Fv fragment such as a disulphide linked Fv or a scFv fragment, or a diabody (all known in the art) derived from such conventional 4-chain antibody, binds to the respective epitope of an antigen by a pair of (associated) immunoglobulin domains such as light and heavy chain variable domains, i.e., by a VH-VL pair of immunoglobulin domains, which jointly bind to an epitope of the respective antigen. An “immunoglobulin single variable domain (ISVD)” as used herein, refers to a protein with an amino acid sequence comprising 4 Framework regions (FR) and 3 complementary determining regions (CDR) according to the format of FR1-CDR1-FR2-CDR2-FR3-CDR3-FR4. An “immunoglobulin domain” refers to “immunoglobulin single variable domains” (abbreviated as “ISVD”), equivalent to the term “single variable domains”, and defines molecules wherein the antigen binding site is present on, and formed by, a single immunoglobulin domain. This sets immunoglobulin single variable domains apart from “conventional” immunoglobulins or their fragments, wherein two immunoglobulin domains, in particular two variable domains, interact to form an antigen binding site. For example, the immunoglobulin single variable domain may be a (single) domain antibody (or an amino acid sequence that is suitable for use as a (single) domain antibody), a “dAb” or dAb (or an amino acid sequence that is suitable for use as a dAb) or a Nanobody (as defined herein, and including but not limited to a VHH); other single variable domains, or any suitable fragment of any one thereof. The term “VHH domain” has been chosen to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”).

The term ‘antibody’ or ‘Fc-fusion’ or ‘VHH-Fc” as used herein further refers to the genetic linking or fusion of antigen-binding fragments or antigen-binding domains with an Fc constant domain as to obtain dimers forming or resembling an antibody structure when expressed in a recombinant host. In particular, antibody fragments, or single domain antibodies such as ISVDs may be C-terminally fused to the N-terminus of an Fc domain, preferably via a linker or hinge region. Alternatively, antibody fragments, or single domain antibodies such as ISVDs, may be fused at the N-terminus to the C-terminal end of an Fc domain, preferably via a linker or hinge region. Said single domain antibody or ISVD fused to said Fc may comprise one or more VHHs or Nbs.

A “therapeutically active agent” or “therapeutically active composition” means any molecule or composition of molecules that has or may have a therapeutic effect (i.e. curative or prophylactic effect) in the context of treatment of a disease. Preferably, a therapeutically active agent is a disease-modifying agent, which can be a cytotoxic agent, such as a toxin, or a cytotoxic drug, or an enzyme capable of converting a prodrug into a cytotoxic drug, or a radionuclide, or a cytotoxic cell, or which can be a non-cytotoxic agent. Even more preferably, a therapeutically active agent has a curative effect on the disease. The composition, or pharmaceutical composition of the invention may act as a therapeutically active agent, when beneficial in treating patients in need for antibody-therapy requiring defucosylated antibodies.

As used herein, the terms “determining,” “measuring,” “assessing,”, “identifying”, “screening”, “addressing”, “testing”, and “assaying” are used interchangeably and include both quantitative and qualitative determinations. “Similar” as used herein, is interchangeable for alike, analogous, comparable, corresponding, and—like or alike, and is meant to have the same or common characteristics, and/or in a quantifiable manner to show comparable results i.e. with a variation of maximum 20%, 10%, more preferably 5%, or even more preferably 1%, or less.

The term “subject”, “individual” or “patient”, used interchangeably herein, relates to any organism such as a vertebrate, particularly any mammal, including both a human and another mammal, for whom diagnosis, therapy or prophylaxis is desired, e.g., an animal such as a rodent, a rabbit, a cow, a sheep, a horse, a dog, a cat, a lama, a pig, or a non-human primate (e.g., a monkey). The rodent may be a mouse, rat, hamster, guinea pig, or chinchilla. In one embodiment, the subject is a human, a rat or a non-human primate. Preferably, the subject is a human. In one embodiment, a subject is a subject with or suspected of having a disease or disorder, or is expected to be at high risk of developing a disease or disorder, in particular a disease or disorder as disclosed herein, also designated “patient” herein. However, it will be understood that the aforementioned terms do not imply that symptoms are present.

The term “enzyme (pre) treatment” or “(pre) treating” or “treat” can be used for in vitro enzymatic treatments, which are excluded form medical purpose and solely indicate that an active enzyme or protein or protein domain is used to perform a chemical reaction on a substrate.

The term “treatment” or “treating” or “treat” can be used in a medical context and is thus defined by a therapeutic intervention that slows, interrupts, arrests, controls, stops, reduces, or reverts the progression or severity of a sign, symptom, disorder, condition, or disease, but does not necessarily involve a total elimination of all disease-related signs, symptoms, conditions, or disorders. Therapeutic treatment is thus designed to treat an illness or to improve a person's health, rather than to prevent an illness. Treatment may also refer to a prophylactic treatment which relates to a medication or a treatment designed and used to prevent a disease from occurring, herein referred to as “prevention”.

A “composition” relates to a combination of one or more active molecules, and may further include buffered solutions and/or solutes such as pH buffering substances, water, saline, physiological salt solutions, glycerol, preservatives, etc. for which a person skilled in the art is aware of the suitability to obtain optimal performance. Suitable conditions as used herein could also refer to suitable binding conditions.

### DETAILED DESCRIPTION

The present invention is based on the finding that a fucosidase enzyme (named ‘H2’ or ‘CC-H2’ further herein) removes core-fucosylation from Fc domains, at least if the N-glycan at N297 is trimmed down to the Man₃ core glycoform, and is optimally active at physiological pH. So in a first aspect, the invention relates to a composition or assembly of components for removing the core-fucose present at the first GlcNac of a N-glycosylated Fc-containing protein, said composition or mixture combining a functional pretreatment enzyme and a GH29 fucosidase enzyme, wherein said fucosidase comprises the CC-H2 sequence, SEQ ID NO: 1, or a functional variant or homologue with at least 85% identity thereof, more preferably at least 95% identity thereof.

With a functional variant or homologue of the GH29 CC-H2 fucosidase is meant herein that the function of being enzymatically active on the same substrate as the wild type CC-H2 fucosidase is maintained in the functional variant or homologous protein, wherein enzymatically active refers to the observation of providing a substrate convergence that is in the same order of magnitude as the wild type enzyme. The functional variant or homologue of the CC-H2 enzyme as provided herein may envisage mutant variants wherein one or more amino acid residues are substituted or mutated to result in a functional variant or homologue with at least 85% amino acid identity to the wild type CC-H2 mature protein form of SEQ ID NO: 1, and thus with retained enzymatic activity on (trimmed) Fc-containing proteins such as N-glycan antibody substrates or N-glycan Fc-based protein substrates. Said substrates may comprise alternative antibody type proteins such as Fc-fusions with antibody fragments or antigen-binding domains, or VHH-Fc fusions, or IgG-type proteins, as described above herein. In a further embodiment, said functional variant or homologue of the CC-H2 fucosidase comprises or consists or an amino acid sequence, in its mature form (i.e. the protein after cleavage of the signal peptide), with at least 50% identity to CC-H2 SEQ ID NO:1, or at least 60% identity to CC-H2 SEQ ID NO:1, or at least 70% identity to CC-H2 SEQ ID NO: 1, or at least 75% identity to CC-H2 SEQ ID NO:1, or at least 80% identity to CC-H2 SEQ ID NO:1, or at least 85% identity to CC-H2 SEQ ID NO:1, or at least 90% identity to CC-H2 SEQ ID NO:1, or at least 95% identity to CC-H2 SEQ ID NO:1, or at least 98% identity to CC-H2 SEQ ID NO:1.

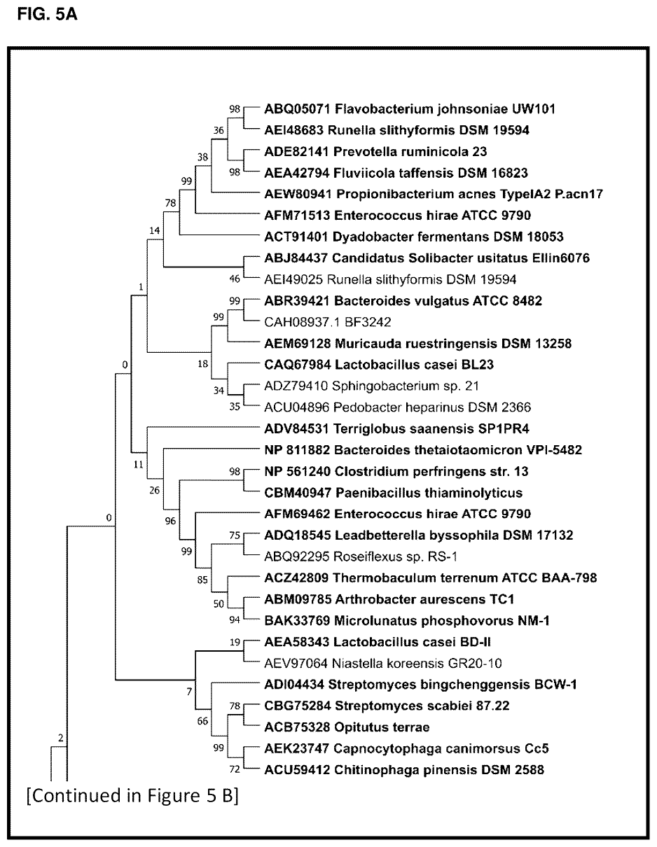

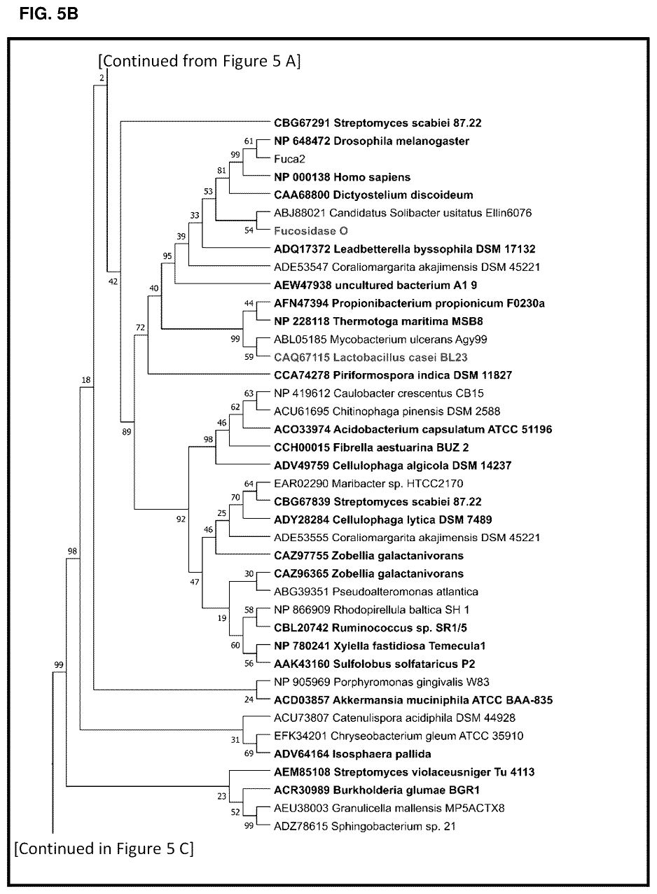

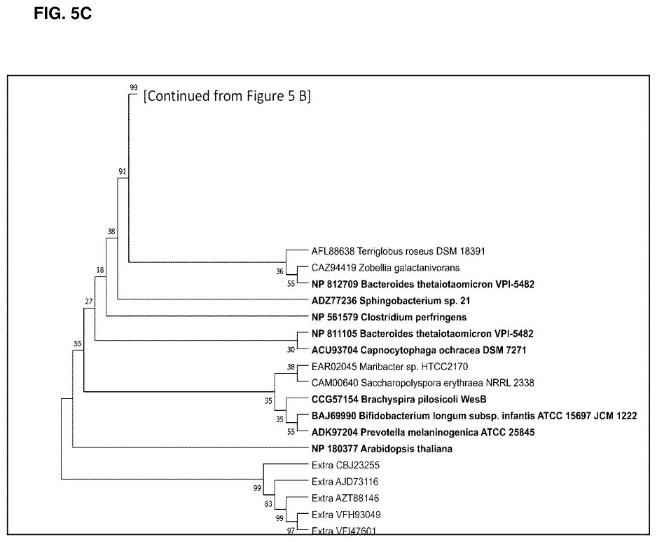

In view of the phylogenetic analysis, it is clear that the CC-GH29 (of which the mature protein form is presented in SEQ ID NO:1) is phylogenetically very different from further fucosidase enzymes with the potential to defucosylate N-glycan antibody substrates, such as Fuc O (FIG. 5). In fact, the closest homologue of CC-H2 of the collection taken up in our analysis was shown to be Chitinophaga pinensis GH29 (FIG. 5A), with an amino acid sequence (of its mature protein form) identity of only 48% to CC-H2 SEQ ID NO:1. Furthermore, a BlastP of the CC-H2 mature protein shows that the naturally-appearing variants within the same species show at least 85% identity to the amino acid sequence of SEQ ID NO:1.

One embodiment relates to said composition in which the pretreatment requires the trimming of naturally intact N-glycans on antibodies to trimannosyl structures, for instance obtained using hexosaminidase thereby providing Man₃GlcNAc₂Fuc-glycans on said Fc-containing proteins as substrate for core-fucosylation by CC-H2. An alternative embodiment relates to said composition in which the pretreatment requires that the CC-H2 is brought in proximity to the N-glycosylated Fc branch, which may be provided by the presence of an ‘N-glycosylated Fc-containing protein scavenger enzyme’, such as catalytically non-active endoglucosaminidases, or domains thereof. So with the term ‘N-glycosylated Fc-containing protein scavenger enzyme’ as used herein is referred to those proteins, in particular inactivated forms of enzymes, which bind the N-glycan structures present on Fc-containing proteins produced in eukaryotic cells, such as endoglucosaminidase enzyme exemplified herein, which are functioning herein as scavengers of the N-glycan antibody substrate for the GH29 fucosidase enzyme, thereby bringing the fucosidase in proximity with the N-glycan substrates. Preferably these scavenger protein domains or enzymes are present in the composition as a fusion with the CC-H2 enzyme or variant of the present invention.

A preferred embodiment relates to said composition, wherein said hexosaminidase comprises the GH20 domain of Endo E (SEQ ID NO: 10 or a homologue with at least 95% identity thereof), and/or said scavenger enzyme comprising an inactive mutant of the GH18 domain of Endo E (SEQ ID NO: 12), or an inactive mutant of EndoS, or EndoS2 (SEQ ID NO: 14, 16, resp.) or a truncated EndoS or EndoS2 enzyme (e.g. as present in SEQ ID NO: 17), or a functional variant or homologue with at least 95% identity thereof.

In a specific embodiment, the combination of the enzymatic activity for defucosylation of N-glycosylated Fc-containing proteins, i.e. the enzymes provided by the GH29 CC-H2 enzyme or functional variant thereof, and the enzyme or enzyme domain or protein domain of one or more pretreatment enzymes, is present as a fusion protein, preferably obtained by recombinant production of a genetic fusion of said enzymes, directly connected, fused by a linker, or designed as swapped or replaced domains of known enzymes. More specifically, examples of fusions as disclosed herein, are provide the fusions of the CC-H2 fucosidase and the GH20 hexosaminidase domain, as exemplified in SEQ ID NOs: 18-19, or a homologue with at least 95% identity of any one thereof, or a fusion of the GH29 CC-H2 and the EndoS or EndoS2 domains, e.g. as in SEQ ID Nos: 20-21.

Alternatively, the invention provides for derived products of said composition, including but not limited to the provision of enzymes or proteins on a solid support structure, such as a resin, column, membrane or coated on a surface, for immobilization; as well as the provision of nucleic acid molecules, vectors, or hosts for recombinantly expressing and producing said composition.

Another aspect of the disclosure relates to a method to produce at least partially defucosylated N-glycosylated Fc-containing proteins, in particular antibodies, comprising the steps of: mixing the protein sample comprising a N-glycosylated Fc-containing protein with said composition described herein, or with the solid support comprising the components of the composition; and isolating the (at least partially) defucosylated N-glycosylated Fc-containing protein from the mixture.

A preferred embodiment discloses the method to produce (at least partially) defucosylated N-glycosylated Fc-containing proteins, comprising the steps of: mixing a protein sample comprising a N-glycosylated Fc-containing protein with the enzymatic composition described herein, or with the solid support comprising the components of the composition described herein, wherein said incubation is performed in physiological conditions, referring to at least in an environment with a neutral pH and at around 37° C.; and isolating the defucosylated N-glycosylated Fc-containing protein from the mixture.

In a further embodiment, the incubation step a. of said method is repeated several times, which means that the N-glycosylated Fc-containing protein sample is initially incubated with an amount of said composition, and after a defined time, additional composition and/or GH29 fucosidase is added to the sample of step a. for further subjecting said N-glycans on said Fc-containing proteins to the enzymatic activity using freshly added enzyme. Alternatively, when the composition or enzymes are present on a solid support, the Fc-containing protein sample may be repeatedly incubated as for step a. by flowing said protein sample from a first enzyme- or composition-containing solid support to a second, third, fourth, and so on, solid support structure or portion containing fresh active enzyme, as to obtain maximal defucosylation of the N-glycans present on the plurality of Fc-containing proteins within the protein sample.

### Glycosyl Hydrolase 29 Family of Alpha-Fucosidase Enzymes

In the present application, the result is described from a search for a fucosidase that could remove the α1-6 linked core-fucose residue from antibodies at their N297 N-glycan, positioned in the Fc domain. Glycosyl hydrolases with an α1-6 core-fucose specificity all cluster together in the GH29 family. The minimal GH29 collection we investigated herein seems to be representative for the diversity of the GH29 family, as indicated in the phylogenetic tree of the complete GH29 family (FIG. 5). Among the fucosidases in our selected collection, only fucosidase H2 (AEK23747; SEQ ID NO:1) from Capnocytophaga canimorsus was found to be able to defucosylate VHH-Fc at its N297 site. This Gram-negative bacterium is a slowly growing commensal found on canine and feline gums. Upon transmission, by mostly bite-wounds, this species can cause illness in humans (Pers et al., 1996. Clinical Infectious Diseases 23:71-75). However, due to its low virulence, healthy people tend to not suffer from illness, in contrast to patients with pre-existing conditions like asplenia, alcoholism and immunosuppression. Additionally, C. canimorsus has been observed to evade the immune system, orchestrated by macrophages, by breaking down their membrane, downregulating production of cytokines such as TNF-α and IL-8, and reducing TLR4 activation. Furthermore, it also evades killing by granulocytes and has been observed to deglycosylate host glycans (Shin et al. 2009. Infection and Immunity 77:2262-2271). In this last context, a fucosidase comes into play. Yet, its activity on core-fucose, still attached to the protein backbone of an antibody, is more striking, as such defucosylation potentiates the antibody which could lead to an increased immune response against the bacterium itself.

Although further enzymes, including closely related ones to CC-H2, showed activity on synthetic substrates, such as CNP-fucose or APTS labeled fucosylated glycans, the activity profile obtained here for CC-H2 on the Fc domain N-glycans was shown to be unique for CC-H2. Furthermore, the conditions, expression levels, and purity of the enzymes used in screening assays also impacts their activity, so potentially alternative enzymes with similar specificity for these natural substrates exist but were not identified herein. From the observation that fucosidase O, the only other fucosidase active on VHH-fc carrying Man₃GlcNAc₂Fuc, and fucosidase H2 do not phylogenetically cluster closely together, neither in our GH29 collection nor in the full GH29 family, we can conclude that antibody defucosylation activity evolved naturally in at least two different clades of the GH29 family, which was not reported or described previously. Hence the identification of this CC-H2 fucosidase having the capacity to defucosylate Fc-tails of antibodies or Fc-containing proteins, was thus unexpected and as demonstrated by the screening and empirical approach determined to be the needle in the haystack of GH29 enzymes known in the public domain.

An approach to screen for additional fucosidase enzymes may however be required, and be based on the presently disclosed screening and selection methods. Successfully generating (large) libraries is usually not the bottleneck in engineering campaigns. However, without a method to screen in a high throughput manner, any engineering would soon become too impractical to manage. In our experiments, we tested the fucosidases first with the simple compound CNP-fucose to explore total activity and subsequently more specifically with our substrate of interest VHH-Fc. In the latter, a rather lengthy protocol is employed to release N-glycans for subsequent labeling and analysis by capillary electrophoresis. Optimizing this protocol could result in a slightly faster workflow. Additionally, a pre-screening step could be introduced to remove any mutant without the desired activity increase, to subsequently test the remaining variants more thoroughly. Such qualitative screening, in which “test-worthy”variants perform better than the current fucosidase, could be achieved by employing agonists that recognize non-fucosylated IgG specifically over fucosylated. Two interactions could allow for such selection. Firstly, we could take advantage of CD16a and its increased binding for non-fucosylated IgG. Yet, with its narrow increase in binding with a factor of 30 between fucosylated and non-fucosylated, fewer variants will be excluded than with a more discriminating binding event. Secondly, we could make use of VHHs selected to specifically recognize non-fucosylated glycoforms of IgG (Kao et al., 2022; PNAS 119 (48) e2212658119). With a synthetic VHH library, the authors screened against antibodies with homogeneous N-glycosylation, obtained chemoenzymatically. Upon affinity maturation, several candidates were disclosed with selective binding to non-fucosylated IgG. Employing one of these VHHs in a pre-screening method could allow to prioritize fucosidase mutants for more in depth analysis of their activities. Additionally, an ELISA type pre-screening would require 20-30× as little VHH-Fc material than our current DSA FACE screen.

### Enhancing the GH29 CC-H2 Enzymatic Activity on N-Glycan Antibody Substrates

As supported by the Examples described herein, C. canimorsus wild type fucosidase, called herein ‘H2’, by itself, or in combination with a hexosaminidase, is capable to defucosylate VHH-Fcs, which is quite unique, though not in a very efficient manner. In our experiments, 2 μg VHH-Fc was incubated with up to 8 μg fucosidase, which taken into account their respective molecular masses of 80 kDa and 50 kDa, indicates that a 6-fold molar excess of enzyme can only generate up to 15% a-fucosylated N-glycans, in the best condition currently identified (ammonium acetate buffer at pH 7). Still, the observation that H2 activity is highest under physiological conditions (pH 7 at 37° C.) is positioning this enzyme as a preferred candidate for further improving the enzyme activity, for instance through engineering and/or fusion of the H2 enzyme.

The experiments revealing the highest levels of defucosylated N-glycans reproducibly are those in which fucosidase H2 is spiked in the reaction at several time points, in ammonium acetate. Yet, at pH 7 this solution is not strongly buffered. Therefore, it would be useful to figure out whether the ammonium or the acetate, or their combination, results in the higher activity. With this knowledge, another buffer system, more compatible with physiological pH, could be tested with NH₄⁺ and/or AcO⁻ as additives. Ideally, the enzyme may be engineered for an increased activity and subsequently optimized conditions can be determined for its engineered variants or mutants, in a similar approach as reported herein for wild type H2.

The expression yield of H2 upon recombinant production in E. coli was suboptimal, so therefore the method for producing said enzyme, may alternatively be applied in more suitable other host systems, for instance using a host from which secretion is easily achieved by including the (naturally occurring) signal peptide of CC-H2, such as Pichia pastoris. Furthermore, fusion to a higher expressed protein such as maltose-binding-protein (MBP) or Small Ubiquitin-like Modifier (SUMO), commonly used also as affinity tag. Interestingly, the EndoE GH20 domain expresses relatively well in E. coli (45 mg/L selective LB), so besides using a composition combining the H2 fucosidase enzyme and a protein or protein domain with hexosaminidase activity, one may recombinantly express a fusion between H2 and EndoE its GH20 domain to improve expression yield, as well as defucosylation, preferably in a synergistic manner. Since EndoE is a dual domain protein by nature, a swap of its natural GH18 domain of 36 kDa for the H2 fucosidase is also envisaged herein as a fusion protein.

Alternatively, similarly to an EndoE fusion, fucosidase H2 enzyme may also be fused with EndoS or Endo S2, two ENGases known to specifically hydrolyze IgG N-glycans located at N297. Swapping their 35 kDa GH18 domains with fucosidase H2 may lead to a properly expressed fusion with improved Fc defucosylation, first of all since full length Endo S2 expresses at 250 mg/L in the currently applied expression systems herein, and since swapped fusions using fucosidases have been reported, for instance by Fan et al. (2023; Biochemical and Biophysical Research Communications, 645, p. 40-46) to provide for a synergistic activity.

An approach to engineer fucosidase H2 for increased activity may be initiated based on the alignment of the fucosidase models and available Fc protein structures, as to design for fucosidase H2 mutant variants, comprising one or more amino acid substitutions for which the activity on VHH-Fc may be tested. One option to provide for an improved activity is to do multiple treatments in smaller amounts of fucosidase H2, so alternatively, an increased stability of the H2 enzyme may be aimed for through engineering.

### Relevance of Defucosylated Antibodies in Increased ADCC Function and Clinical Utility

We tested fucosidase H2 on VHH-Fc pretreated with EndoE GH20, which removed the terminal GlcNAc residues resulting in the Fc domain carrying Man₃GlcNAc₂Fuc. While this substrate was (partially) defucosylated by the enzyme, the further aim is to defucosylate antibodies rather than VHH-Fc molecules, as antibodies are still the most relevant glycoform for clinical purposes. The present application provides for two VHH-Fc proteins as substrates, which however both carried wild type IgG1 Fc domains, so plausibly positively translating fucosidase activity to fully human IgG1 antibodies.

Moreover, the relevance of the trimmed trimannosyl version is arguable, as it is known that this results in increased clearance rates because of interactions of the terminal mannose residue with mannose receptors on macrophages and liver cells. Additionally, the level of ADCC capacity with antibodies carrying this shorter Man₃GlcNAC₂ N-glycan needs to be verified. By means of chemoenzymatic workflows, antibodies with homogeneous non-fucosylated glycoforms have been created (Kurogochi et al. 2015; PLOS ONE 10(7): e0132848). By varying the oxazoline-activated donor glycan, the resulting glycoforms ranged from the largest complex type (Sia₂Gal₂GlcNAC₂Man₃GlcNAC₂) to the Man₃GlcNAc₂. Kurogochi et al. (2015, supra) show that the affinity for CD16a of trastuzumab-Man₃GlcNAc₂ is the lowest among the chemoenzymatically modified variants, at the same level of fucosylated CHO produced IgG. Yet, when comparing cell killing through ADCC, Man₃GlcNAc₂ outperformed fucosylated IgG, making its activity more similar to GlcNAC₂Man₃GlcNAC₂.

In a further aspect of the invention, the method for producing defucosylated N-glycosylated Immunoglobulin G1 (IgG1) antibodies, comprises the steps of:
a. Incubating a protein sample in vitro with the composition described herein, wherein said protein sample may be a sample obtained from a patient or a component or fraction thereof, containing human IgG1s, even more preferably a sample obtained by plasmapheresis;
b. Isolating the IgGs from said sample incubated with said composition, preferably using IgG capturing methods known in the art, or alternatively, by collecting the flow-through when the enzyme composition is provided on a solid support.

A further application of the invention provides for a third aspect which relates to the (at least partially) defucosylated IgG-containing sample obtained from or obtainable by said method. A further embodiment relates to said (at least partially) defucosylated IgG-containing sample obtained from or obtainable by said method, for use in therapy, or more specifically, for use to treat a subject in need of (at least partially) defucosylated IgGs, preferably for use in treatment wherein treatment is performed by re-infusion of said sample resulting from the defucosylation method.

An alternative application of the invention provides for a further aspect which relates to the method of producing (at least partially) defucosylated N-glycan-containing antibodies, comprising the steps of:
a. Incubating a protein sample, which comprises recombinantly produced N-glycosylated antibodies, preferably produced in an eukaryotic host, such as a mammal or human, with the composition or solid structure as described herein, and
b. Isolating or purifying the (at least partially) defucosylated N-glycan IgGs or antibodies from said mixture.

So in a specific embodiment the sample subjected in step a. of said method is the resulting product of a manufactured therapeutic antibody recombinantly produced in a mammalian host, such as a CHO cell culture, containing antibodies with an N-glycan structure with a core fucose. As described previously, the removal of said fucose increases the potency of said therapeutic antibodies in several applications, such as anti-tumor treatment where an increase in ADCC effector function is expected, potentiating recombinant antibodies, more specifically monoclonal antibodies against cancer.

So in a further specific embodiment, said (at least partially) defucosylated antibodies obtained from the enzyme treatment or incubation with the composition described herein may be used for treating a patient, wherein said initial antibodies subjected to defucosylation are patient-derived antibodies, thus resulting in the treatment of said patient with its own antibodies, in a-fucosylated form. Indeed, one ultimate goal is thus to defucosylate patient-derived antibody samples. Tumor-infiltrating B lymphocytes may produce anti-tumor antibodies of the IgG1 subclass (Sharonov, et al. 2020; Nat Rev Immunol 20, 294-307). In the assumption that some of these antibodies end up in circulation, activating them by removing their core-fucose is in scope. For instance, if (a fraction of) the total antibody pool could be treated during plasmapheresis, they would re-enter the patient's body defucosylated, plausibly ADCC-activated, potentially resulting in improved recruitment of NK cells. One advantage of the latter application is that there is no requirement to know which antigen(s) triggered the patient's immune system, but by potentiating their resulting antibodies, to overcome the tumor's immunosuppressive mechanisms that render these endogenous anti-tumor antibodies incompetent in clearing the tumor.

It is to be understood that although particular embodiments, specific configurations as well as materials and/or molecules, have been discussed herein for methods, and products according to the disclosure, various changes or modifications in form and detail may be made without departing from the scope of this invention. The following examples are provided to better illustrate particular embodiments, and they should not be considered limiting the application. The application is limited only by the claims.

### EXAMPLES

### Example 1. Selection of GH29 Family Members for Testing Defucosylation Activity on Natural Fcs of Antibodies

Through whole genome sequencing and in-depth analyses of bacterial genomes, the GH29 fucosidase family has been established from about 600 sequences about 10 years ago, to a close to a 10.000 genes nowadays, all clustering together based on their amino acid sequences. Initially, bacteria with known or annotated fucosidase genes were cultivated to subsequently test their enzymatic activity on simple chromogenic substrates, with only limited activity detectable in few bacterial strains. In order to increase the selection of active enzymes, the strategy shifted to recombinant production of the fucosidases. To lower the cost and work, while maintaining maximal diversity with the least amount of recombinant proteins, the GH29 family members were clustered phylogenetically in order to prune the collection based on the length of loops surrounding the conserved catalytic residues. Based on this clustering activity, we selected a collection of 90 sequences, largely from bacterial origin with a few eukaryotic and archaeal genes. Upon cloning and bacterial intracellular expression, lysates and purified fucosidases were tested on a variety of fucosylated substrates. However, none of the enzymes tested showed activity on core-fucosylated antibodies from commercial human sera.

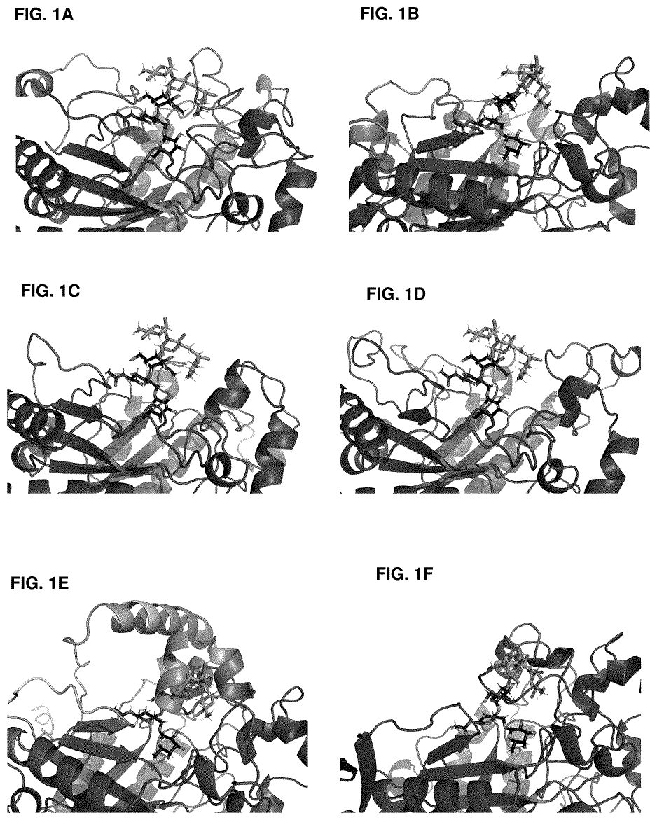

Next, AlphaFold2 models were generated for a downsized-GH29 collection to search for enzymes with a minimally constrained catalytic site to accommodate both the Fc backbone and complex N-glycans. For several proteins, the active site was positioned closely to the surface in the generated model. FIG. 1 shows a few examples of such conformations (AlfC, fucosidase O, A8 and H2 enzymes, as described herein) and examples of enzymes with a more closed structure near the active site (A2 and C9 enzymes). We therefore prioritized the enzymes with an open active site for screening for activity on VHH-Fc proteins produced in CHO cells.

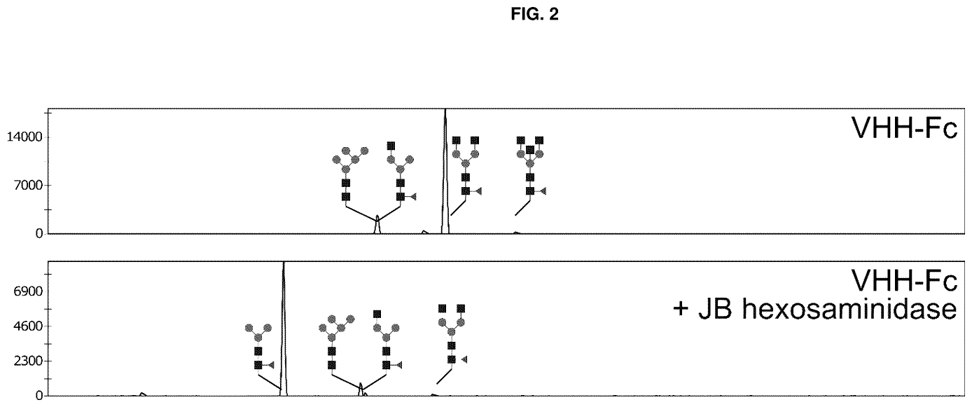

To test defucosylation activity of these enzymes on Fc domains, we initially opted to use VHH-Fc proteins produced in CHO cells, which mainly carry GlcNAc₂Man₃GlcNAc₂Fuc. However, based on structural alignments of fucosidases such as AlfC with docked N-glycans, we decided to test defucosylation on VHH-Fc with shorter N-glycans which may obstruct larger N-glycans to fit in for catalysis (FIG. 1). Even though loops are generally mobile, especially near the surface of the protein, their overall location mostly does not change drastically and, additionally, such induced fit catalysis is often correlated with slower kinetics. We hypothesized that by employing a VHH-Fc with smaller N-glycans as substrate, we would increase the likelihood of finding active enzymes in our screening. To obtain VHH-Fc carrying terminal mannose residues, we treated VHH-Fc with hexosaminidase enzymes to remove the terminal GlcNAc residues, resulting in the shorter Man₃GlcNAc₂Fuc N-glycan. We successfully trimmed down the terminal GlcNAc residues of intact VHH-Fc with commercial hexosaminidase extracted from Jack Beans. Before hexosaminidase digest, the main N-glycan is GlcNAC₂ Man₃GlcNAC₂ Fuc, with a minimal amount of GlcNAcMan₃GlcNAc₂Fuc, while this shifts after digestion to a population consisting mainly of Man₃GlcNAc₂Fuc with a limited amount of GlcNAcMan₃GlcNAC₂Fuc (FIG. 2).

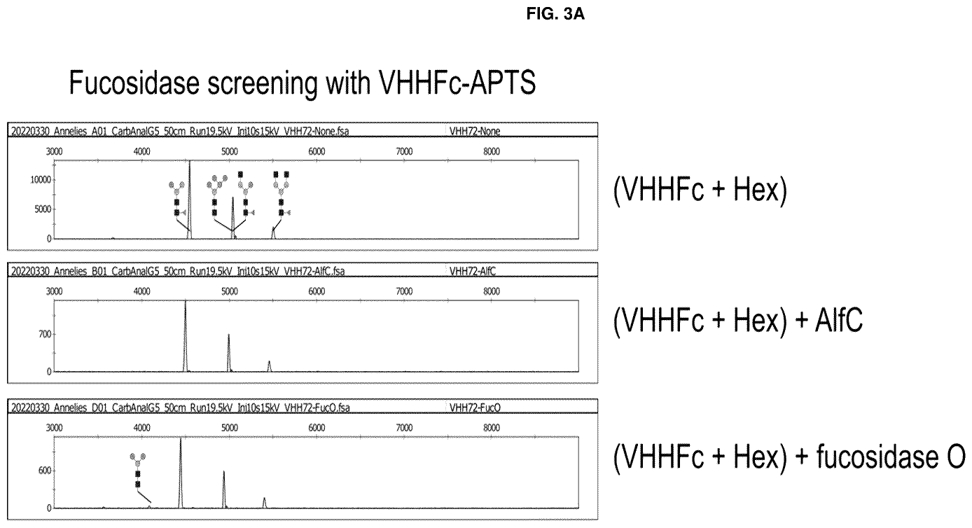

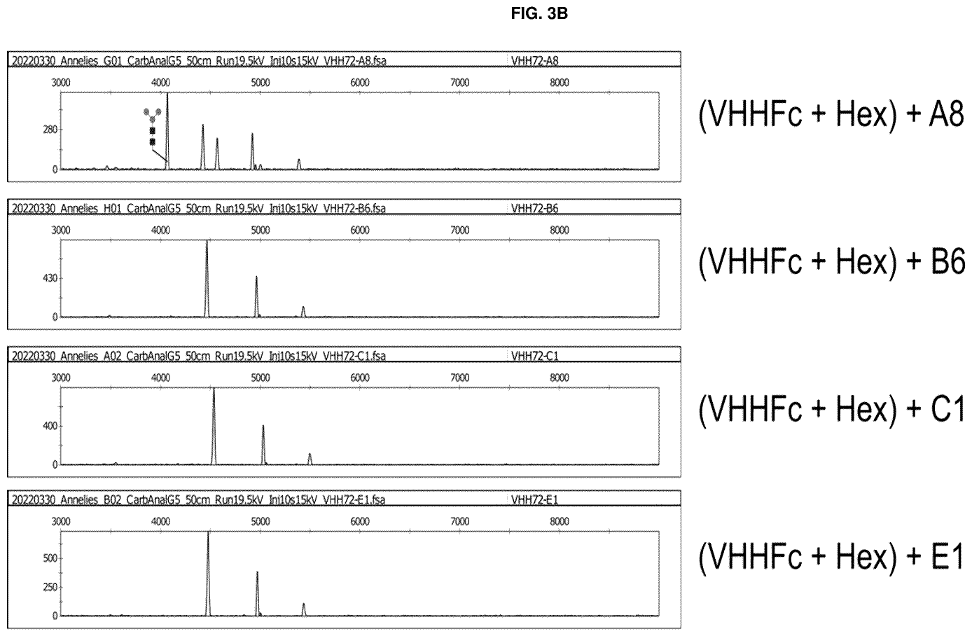

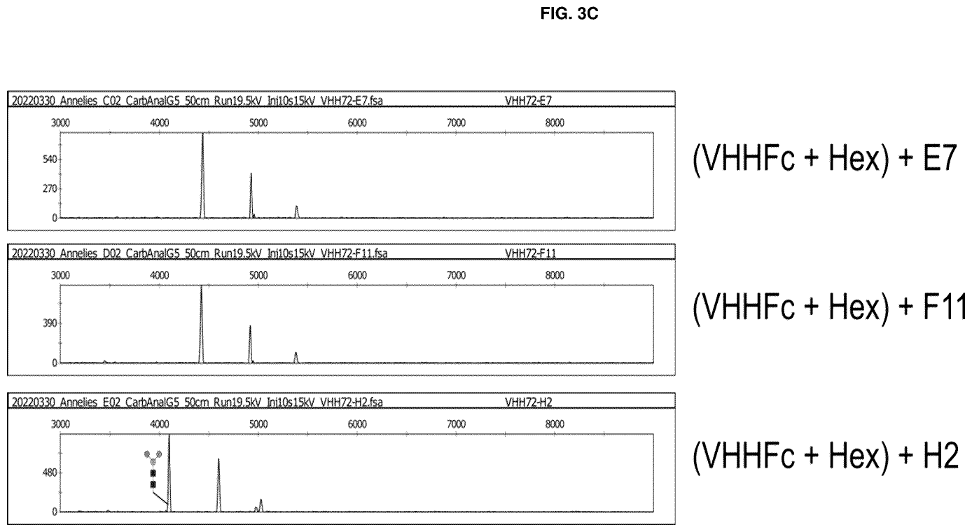

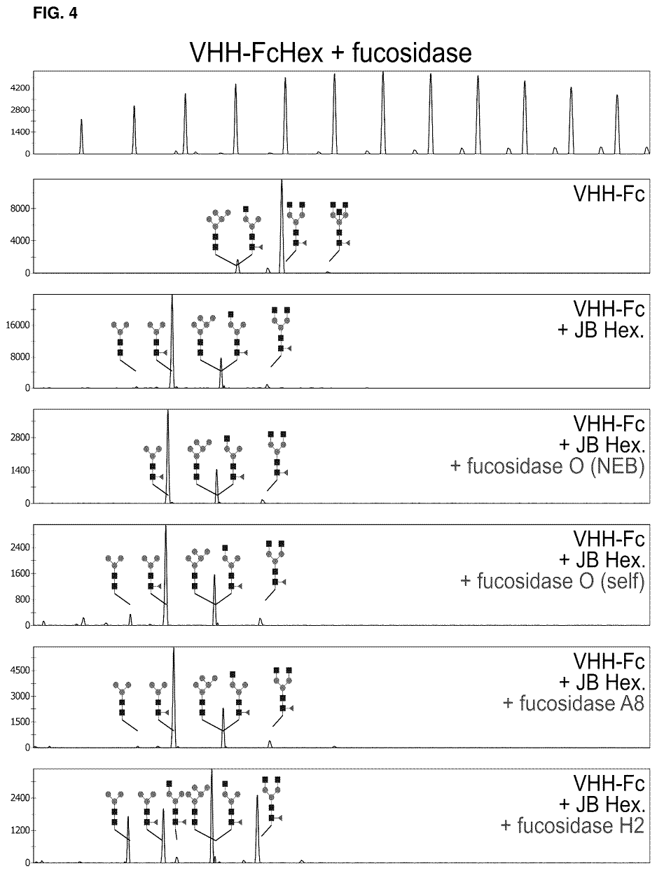

We subsequently used VHH-Fc, pretreated overnight with hexosaminidase, as a substrate to test GH29 enzymes with an open active site. These enzymes (as named herein: A8, B6, C1, E1, E7, F11 and H2) were expressed in E. coli BL21-AI cells at small scale for gravity flow Ni-NTA purification. This crude purification allowed us to quickly test the fucosidases on APTS-labeled N-glycans originating from VHH-Fc, treated with Jack Bean hexosaminidase. In the resulting DSA FACE profiles (FIG. 3), we observed defucosylation of APTS-labeled N-glycans for three enzymes at acidic pH (NH₄AcO— pH 5): fucosidase O (in-house produced), A8 and H2, although only for the latter enzyme complete defucosylation was observed.

Upon demonstrating that A8 and H2 enzymes were capable of removing core-fucosylation from APTS labeled N-glycans, we decided to repeat their expression, next to fucosidase O as a benchmark, on a larger scale and purify them with an ÄKTA system over IMAC and SEC columns to obtain fucosidases with a higher purity. We next incubated the fucosidases with VHH-Fc, pretreated overnight with Jack Bean hexosaminidase at 37° C., in NEB glycobuffer 1, a buffer indicated by NEB for optimal activity of fucosidase O (pH 5.5), which was included as a benchmark. We concluded that fucosidase A8 (P. thiaminolyticus) and fucosidase O from NEB have no activity, while fucosidase O produced in house and fucosidase H2 (C. canimorsus, SEQ ID NO:1) are active on Man₃GlcNAC₂Fuc (Error! Reference source not found. 4), though fucosidase O only to a limited extent. Fucosidase H2 generates the most non-fucosylated N-glycans in the tested condition: Man₃GlcNAc₂ takes up 16% in the total pool. Considering the 20% relative intensity of leftover Man₃GlcNAc₂ Fuc in the total pool, this translated to a defucosylation of 45% of its possible substrate. Additionally, some non-fucosylated monoGlcNAcylated N-glycan popped up after digest with fucosidase H2, while larger N-glycans have not been defucosylated.

### Example 2. Further Screening of the GH29 Enzyme Collection for Activity on VHH-Fcs

Motivated by the successful defucosylation of intact VHH-Fc, we set out to further test our GH29 collection for any enzymes active to a similar extent as fucosidase H2 from C. canimorsus. By identifying additional fucosidases active on the Fc-fold, further insights in how to narrow down which amino acids are important for activity may be obtained, and could indicate which phylogenetic branch(es) of the GH29 family show(s) activity on glycosylated Fc-containing protein substrates.

In previous work we tested the protein expression yield of the GH29 collection and found that only 50% surpassed an arbitrarily, lowly set limit of 1 mg protein yield from 1 L culture volume. In order to prioritize which enzymes to express first, and to include all close relatives of H2 even if their expression is below the limit, we opted to construct a bootstrapped phylogenetic tree. Using the MEGA11 software, a multiple sequence alignment (MSA) was created (Muscle algorithm) from the GH29 collection expanded with fucosidase O, as it is our benchmark for (partial) activity on Fc-containing proteins, and a group of fucosidases from GH95, which were expected to cluster together but separate from the main collection. Because of this extra group the phylogenetic tree could be rooted, allowing us to correctly determine the diversification distance between the GH29 sequences. Subsequently, the substitution model that best described the MSA with the fewest parameters, was inferred from the data to be WAG+G, an approximate maximum likelihood method which assumes the substitution rate to vary from site to site within a sequence. Finally, with these parameters (WAG+G) a maximum likelihood based phylogenetic tree was built iteratively 1000×. Such a bootstrapped tree allows to quantify the likelihood that branches cluster together solely by chance.

From the phylogenetic tree, shown in FIG. 5 we inferred that a group of sequences clusters together with C. canimorsus fucosidase (AEK23747), also called ‘H2’ or ‘CC-H2’ herein, from: AEA58343, ACU59412, ADI04434, CBG75284 and ACB75328, noted respectively as A4, A9, A11, A12 and H3 herein during expression and activity screening (Table 1). These close relatives, if active on the VHH-Fc substrate, could be vital in discovering key features in the sequence of defucosylation of intact VHH-Fc with complex N-glycans. The importance of performing this phylogenetic check of our collection becomes evident when combining the expression data for the close relatives. Based on expression yield, only A11 would have qualified for testing (http://hdl.handle.net/1854/LU-8549769: Grootaert H. (2017, PhD, University of Gent, Belgium: Exploring glyco-active enzymes for biomedical glycan manipulation). The final set of sequences to test for defucosylation of intact antibodies contained 61 fucosidases, covering the or GH29 collection quite generally with members being tested from each clade (FIG. 5). Fucosidases were expressed intracellularly in E. coli BL21-AI cells. The volume for expression ranged from 50 ml to 3 L to ensure that minimally 0.5 mg fucosidase was obtained after purification. After overnight expression, cells were sonicated and the resulting lysates were clarified for loading onto a HisTrap column for IMAC based purification of the His-tagged fucosidases. The fractions after size exclusion chromatography (SEC) that contained active fucosidase, based on activity on synthetic substrate (2-chloro-4-nitro-phenol fucose, CNP-fucose), were pooled and frozen in liquid nitrogen until further analysis.

TABLE 1
Overview of the GH29 fucosidase present in our collection.

| | GenBank | |
|---|---|---|
| ID | identifier | Organism (strain) |
| A01 | CAQ67984 | Lactobacillus casei |
| A02 | CAQ67115 | Lactobacillus casei |
| A03 | ADV64164 | Isosphaera pallida |
| A04 | AEA58343 | Lactobacillus casei |
| A05 | AEM69128 | Muricauda ruestringensis |
| A06 | ABR39421 | Bacteroides vulgatus |
| A07 | ADQ18545 | Leadbetterella byssophila |
| A08 | CBM40947 | Paenibacillus thiaminolyticus |
| A09 | ACU59412 | Chitinophaga pinensis |
| A10 | ABQ92295 | Roseiflexus sp. |
| A11 | ADI04434 | Streptomyces bingchenggensis |
| A12 | CBG75284 | Streptomyces scabiei |
| B01 | ACT91401 | Dyadobacter fermentans |
| B02 | ADQ17372 | Leadbetterella byssophila |
| B03 | AFL88638 | Terriglobus roseus |
| B04 | ACZ42809 | Thermobaculum terrenum |
| B06 | ABM09785 | Arthrobacter aurescens |
| B07 | ADE82141 | Prevotella ruminicola |
| B08 | NP_000138 | Homo sapiens (coli codon |
| | | optimization) |
| B09 | NP_000138 | Homo sapiens (no codon |
| | | optimization) |
| B10 | NP_561579 | Clostridium perfringens |
| B11 | CAZ97755 | Zobellia galactanivorans |
| B12 | AEW47938 | uncultured bacterium |
| C01 | AFM69462 | Enterococcus hirae |
| C02 | AFN47394 | Propionibacterium propionicum |
| C03 | NP_561240 | Clostridium perfringens |
| C04 | AEM85108 | Streptomyces violaceusniger |
| C05 | BAK33769 | Microlunatus phosphovorus |
| C06 | NP_812709 | Bacteroides thetaiotaomicron |
| C07 | CAA68800 | Dictyostelium discoideum |
| C08 | AFM71513 | Enterococcus hirae |
| C09 | NP_228118 | Thermotoga maritima |
| C10 | ABJ84437 | Candidatus Solibacter usitatus |
| C11 | AEW80941 | Propionibacterium acnes |
| C12 | NP_811882 | Bacteroides thetaiotaomicron |
| D01 | AEA42794 | Fluviicola taffensis |
| D02 | AEI48683 | Runella slithyformis |
| D03 | NP_811105 | Bacteroides thetaiotaomicron |
| D05 | ABJ88021 | Candidatus Solibacter usitatus |
| D06 | NP_648472 | Drosophila melanogaster |
| D07 | ADE53555 | Coraliomargarita akajimensis |
| D08 | ABQ05071 | Flavobacterium johnsoniae |
| D09 | ADY28284 | Cellulophaga lytica |
| D10 | CAZ96365 | Zobellia galactanivorans |
| D11 | BAJ69990 | Bifidobacterium longum subsp. |
| | | infantis |
| D12 | AEU38003 | Granulicella mallensis |
| E01 | CCG57154 | Brachyspira pilosicoli |
| E02 | ADZ78615 | Sphingobacterium sp. |
| E03 | ADV49759 | Cellulophaga algicola |
| E04 | AAK43160 | Sulfolobus solfataricus |
| E05 | ADZ79410 | Sphingobacterium sp. |
| E06 | NP_180377 | Arabidopsis thaliana |
| E07 | ADK97204 | Prevotella melaninogenica |
| E08 | ABL05185 | Mycobacterium ulcerans |
| E09 | AEV97064 | Niastella koreensis |
| E10 | NP_419612 | Caulobacter crescentus |
| E11 | ABG39351 | Pseudoalteromonas atlantica |
| E12 | EAR02290 | Maribacter sp. |
| F01 | ACD03857 | Akkermansia muciniphila |
| F02 | ACU61695 | Chitinophaga pinensis |
| F03 | NP_780241 | Xylella fastidiosa |
| F04 | AEI49025 | Runella slithyformis |
| F05 | CAM00640 | Saccharopolyspora erythraea |
| F06 | CCH00015 | Fibrella aestuarina |
| F07 | ACO33974 | Acidobacterium capsulatum |
| F08 | ADE53547 | Coraliomargarita akajimensis |
| F09 | ACU04896 | Pedobacter heparinus |
| F10 | ACU73807 | Catenulispora acidiphila |
| F11 | ADZ77236 | Sphingobacterium sp. |
| F12 | NP_866909 | Rhodopirellula baltica |
| G01 | CAZ94419 | Zobellia galactanivorans |
| G03 | CBG67291 | Streptomyces scabiei |
| G04 | NP_905969 | Porphyromonas gingivalis |
| G05 | CBG67839 | Streptomyces scabiei |
| G06 | EAR02045 | Maribacter sp. |
| G09 | EFK34201 | Chryseobacterium gleum |
| H01 | ADV84531 | Terriglobus saanensis |
| H02 | AEK23747 | Capnocytophaga canimorsus |
| H03 | ACB75328 | Opitutus terrae |
| H04 | CBL20742 | Ruminococcus sp. |
| H05 | ACR30989 | Burkholderia glumae |
| H06 | ACU93704 | Capnocytophaga ochracea |
| H07 | CCA74278 | Piriformospora indica |

GenBank identifiers are given, next to short ID numbering introduced for simplification in the examples and application as used herein, and organism of origin.

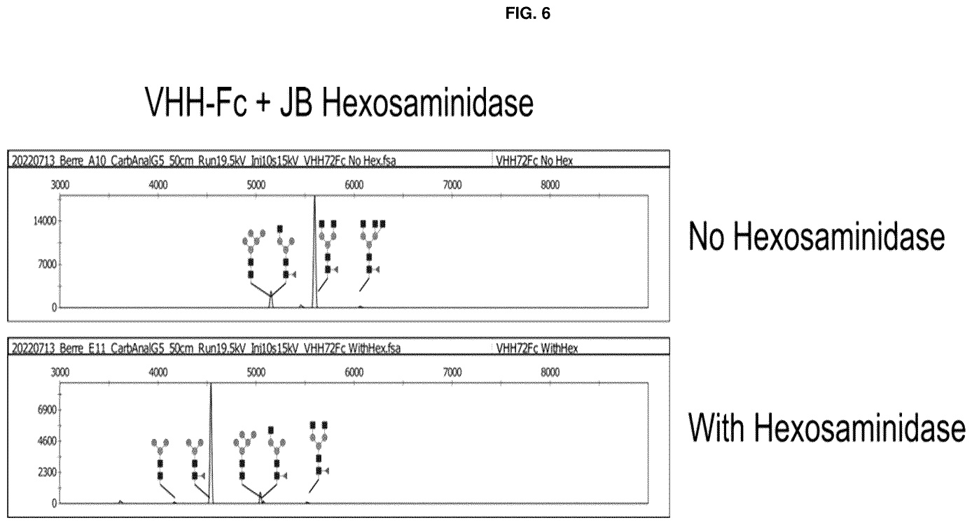

To generate sufficient VHH-Fc carrying Man₃GlcNAc₂ Fuc, we decided to shift from the commercial Jack Bean hexosaminidase to a less expensive, recombinant solution. EndoE, a multi-modular glycosyl hydrolase from Enterococcus faecalis, contains a GH20 domain with an activity as exo-β-1,2-N-acetylglucosaminidase, alongside a GH18 domain that functions as an endo-N-acetylglucosaminidase (ENGase). Interestingly, the GH20 domain was verified to remove GlcNAc residues from fully intact IgG (García-Alija, M., Du, J. J., Ordóñez, I. et al. 2022; Nat Commun 13, 1137), making it the ideal substitute for the commercial Jack Bean enzyme. Upon successful expression of the GH20 domain of Endo E in E. coli BL21-AI cells, we confirmed its exoglycosidase activity on CHO produced VHH-Fc (FIG. 6).

We subsequently generated a fucosidase screening substrate by treating VHH-Fc (carrying 88% GlcNAC₂Man₃GlcNAc₂Fuc with 12% GlcNAcMan₃GlcNAc₂Fuc) with Endo E GH20 for 24 h at 37° C. in PBS. After a protein A chromatographic cleanup, the final product contained 38% Man₃GlcNAc₂Fuc, 60% GlcNAcMan₃GlcNAC₂Fuc and 2% GlcNAc₂ Man₃GlcNAc₂Fuc (FIG. 6B). Higher productivity of the Endo E GH20 domain could have been obtained by optimizing the conditions, yet this was not included as the heterogeneity of this substrate would allow its use in subsequent steps in our research line. Initially, activity on Man₃GlcNAC₂Fuc is tested.

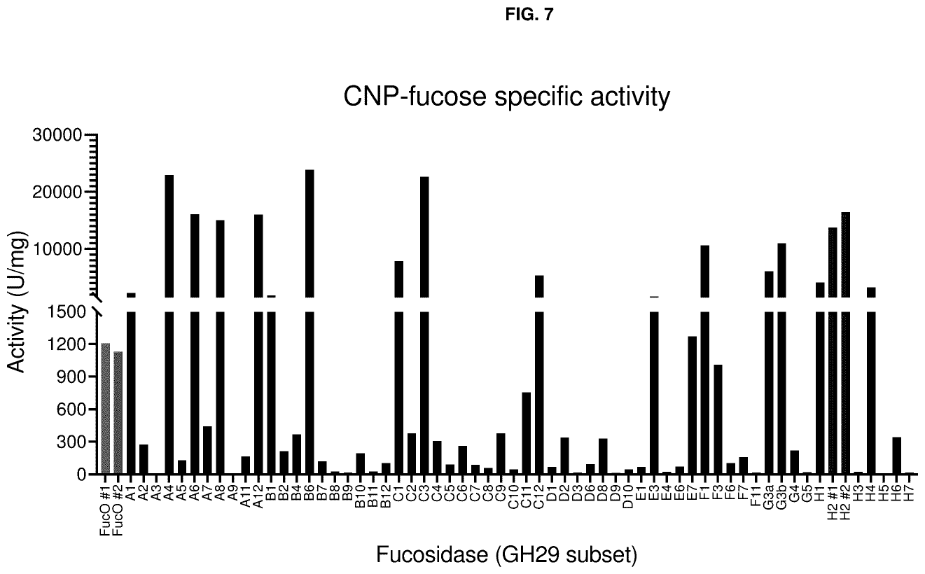

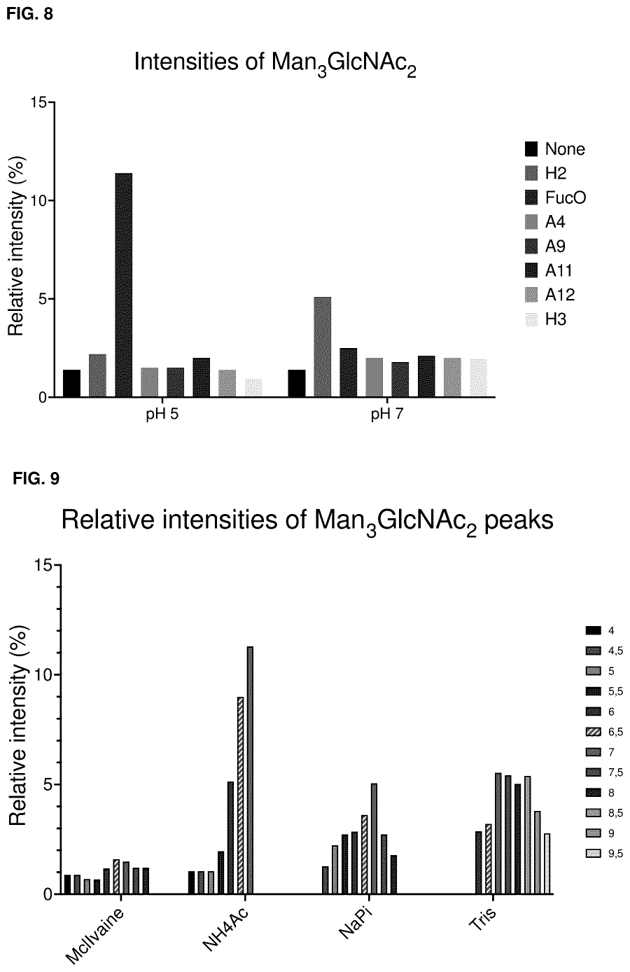

Having generated sufficient fucosylated VHH-Fc without GlcNAc residues, the produced fucosidases from the GH29 collection were tested. However, despite showing (mostly limited) activity on synthetic CNP-fucose (FIG. 7) at pH 7, none of the expressed fucosidases from the collection, aside from fucosidase O and H2, could remove core-fucosylation in the tested conditions at acidic or physiological pH. FIG. 8 shows the percentage of detected Man₃GlcNAc₂ peak in DSA FACE profiles for the close relatives of H2 (A4, A9, A11, A12 and H3) in the defucosylation screening at pH 5 and pH 7 besides H2 and Fuc O. At neither pH conditions does the non-fucosylated Man₃GlcNAc₂ peak increase relative to the level already present in the VHH-Fc condition without fucosidase treatment for the ‘phylogenetically close relatives of H2’. So based on their phylogenetic relationship, it is not possible to cluster the active fucosidases for this substrate class.

In conclusion, the CC-H2 GH29 enzyme seems to have its optimal activity under physiological conditions (pH7) (FIG. 9), and provides for a valuable candidate to further develop for efficient defucosylation of intact antibodies (optionally pretreated to trim down the Man₃GlcNac₂Fuc). Moreover, the fact that physiological conditions are applicable when using CC-H2 in enzymatic treatments of antibodies is of advantage for use in medical workflows and/or cellular conditions.

### Example 3. Optimizing the Conditions for Activity of Fucosidase H2

Despite fucosidase CC-H2 showing activity on intact VHH-Fc carrying Man₃GlcNAc₂Fuc, the levels of non-fucosylated N-glycan after overnight incubation are rather low, never contributing more than a few percent to the total pool (FIG. 8). In order to increase productivity, several conditions were screened.

Firstly, we assessed the effect of pH on activity of fucosidase H2 during overnight incubations at 37° C. Since buffering capacity at different pH values varies among buffers, we tested several buffers with a range of pH conditions. The VHH-Fc was analyzed for N-glycan composition by DSA FACE after the overnight incubation. For simplicity, the data is represented in FIG. 9 as the percentages of Man₃GlcNAc₂ generated. Fucosidase H2 is most active at physiological pH, as exemplified by the activity in the buffers ammonium acetate, sodium phosphate and Tris, while little to no activity could be detected in the Mcllvaine buffer (see Experimental procedures). Possibly, the chelating characteristic of citric acid may be to blame. Among the buffers permitting defucosylation, the ammonium acetate buffer performs best, with approximately twice as potent activity compared to phosphate and Tris based conditions.

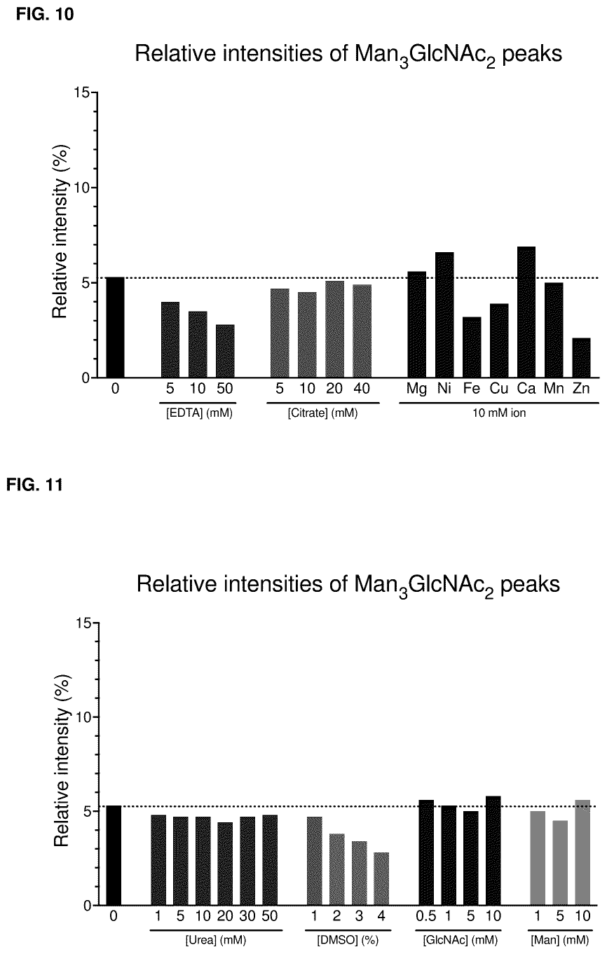

The lack of activity in Mcllvaine prompted us to test whether the chelating potential of citrate could be the cause. In the Mollvaine buffer system, 0.1M citrate acid and 0.2M Na₂HPO₄, are used to vary the pH from 3.0 to 8.0. Citric acid is a known chelator which could impact activity of the fucosidase, so we attempted to test the effect of different citrate concentrations on defucosylation by fucosidase H2. We incubated VHH-Fc with fucosidase H2 with different concentrations of citrate in Na₂HPO₄ buffer, set at pH 7. Moreover, to test whether the chelating aspect of citrate could be causing loss of enzymatic activity, we tested the addition of another chelating component ethylenediaminetetraacetic acid (EDTA) in parallel. After overnight incubation at 37° C., we analyzed the N-glycans released from VHH-Fc (FIG. 10). EDTA appears to have an inhibitory effect on the activity of fucosidase H2, while for citrate there seems to be no impact, though the activity of the fucosidase enzyme was limited to begin with. Additionally, we also tested the effect of EDTA and citrate on the general activity of fucosidase H2 with CNP-fucose instead of VHH-Fc as substrate. Yet, limited effect could be detected. Possibly the requirement for bivalent ions, which can be chelated by EDTA and citrate, is different for small molecule substrate than for fucosylated IgG.

To investigate potential effects of bivalent ions, we incubated seven different bivalent ions (Mg²⁺, Ni²⁺, Fe²⁺, Cu²⁺, Ca²⁺, Mn²⁺, Zn²⁺; all as SO₄²⁻ salts) with fucosidase H2 and VHH-Fc (FIG. 10). Mg²⁺ and Mn²⁺ did not impact activity of fucosidase H2, while Fe²⁺, Cu²⁺ and Zn²⁺ decrease the extent of defucosylation. Intriguingly, the activity is slightly boosted in Ni²⁺ and Ca²⁺.

Next to testing whether chelators and bivalent ions influence the overall activity of fucosidase H2, we decided to verify whether structurally interfering with the Fc fold could increase defucosylation. There is evidence that the N297 containing peptide loop may infrequently move away from the protein backbone (Lee, and Im, 2017; Sci Rep 7, 12659), making it more accessible at the glycan termini for modification and possibly as well for defucosylation. As such, we tested the impact of chaotropes on defucosylation. We incubated VHH-Fc with fucosidase H2 in the presence of limited amounts of urea and DMSO, to subtly disturb protein fold. Additionally, the N-glycan situated at N297 is known to interact with the protein backbone via its α1-6 arm. Hypothesizing that improved defucosylation could be attained by weakening this interaction, we attempted to test the H2 enzyme in the presence of GlcNAc and mannose (FIG. 11). Activity of fucosidase H2 is only impacted by DMSO, yet negatively. Likely this reduction in activity is due to the fucosidase losing structural stability in increasing amounts of DMSO, as indicated by a loss of activity with synthetic substrate CNP-fucose.

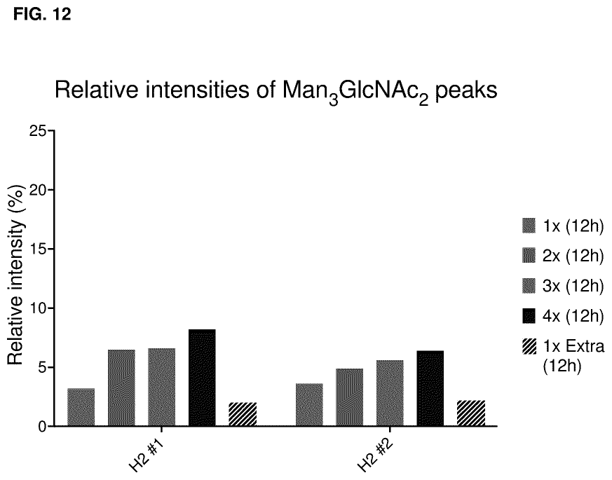

In order to increase the hydrolysis of core-fucoses in VHH-Fc, we attempted repeatedly adding the enzyme to boost the fraction of Man₃GlcNAC₂ in the total pool. We explored this concept by testing different incubation durations (5 h versus overnight) and by repeatedly adding more fucosidase (up to 4×) to already incubated samples. A condition (FIG. 12: 1×‘Extra’) was included in which 8 μg fucosidase was added at once for a single overnight incubation, identical to the condition in which 2 μg fucosidase is added four times, except for the timing as the multiple addition required longer incubation. Two experiments were performed with 2 differently prepared recombinant purified H2 batches (#1 and #2), and we observed that the fraction non-fucosylated glycans increased when enzyme was added multiple times (FIG. 12). When comparing the results of the 4× boosted 2 μg condition with the single boosted 8 μg condition, a difference can be noted (FIG. 12), wherein the level of defucosylation is highest for the multiple additions of enzyme, which indicates that the enzyme may lose activity during extended incubation times. A side-by side experiment also allowed to compare the activities of the two batches of fucosidase H2, from which it became clear that the second batch H2 #2 is slightly less active as compared to the first.

### Example 4. Fusion of H2 Fucosidase to Broaden and Increase its Activity on N-Glycosylated Fc-Containing Proteins

To provide for a single protein which is capable to defucosylate intact N-glycosylated Fc-containing proteins, in particular antibodies, the wild type or variant H2 fucosidase (SEQ ID NO:1) may be fused to other glycosyl hydrolase (GH)-domains, or GH-containing enzymes to boost the activity on antibody core-fucose residues.

For instance, Endo-β-N-acetylglucosaminidase E or in short ‘Endo E’ contains two GH domains: GH18, an endoglucosaminidase (ENGase), and GH20, a hexosaminidase. The GH20 domain was already verified to hydrolyze terminal GlcNAc residues of IgG, which is required for activity of its GH18 domain on the resulting deGlcNAcylated N-glycan; EndoS and EndoS2 are two 90 kDa GH18 containing enzymes, with a strict preference of IgG as substrate. Next to GH18 domains, they harbor LRR domains and CBM domains, of which the latter is verified to impart the specificity.

So, boosting fucosidase H2's activity on Fcs may be done by combining the different proteins, to act in a synergistic manner on the N-glycan of the Fc-containing protein, though the ENGase domains should be removed or inactivated by mutations as to avoid trimming to fucosylated GlcNAc residue which would result in a structure of the Fc domain that is similarly collapsed as non-glycosylated IgG, thereby reducing effector functionality. Such catalytically inactivated mutant GH18 Endo E is provided in SEQ ID NO: 12, and inactivated mutant EndoS and EndoS in SEQ ID NO:16 and 17, resp. The H2 fucosidase and mutant ENGase may be provided as a composition comprising both proteins, or alternatively, the fucosidase may be fused genetically to the NGase, such as EndoE, EndoS or EndoS2. For EndoE, two options exist: we either link the fucosidase enzyme directly to the GH20 domain (SEQ ID NO: 18), or we swap the GH18 domain for the fucosidase enzyme (SEQ ID NO: 19). The latter fusion would be 12 kDa larger, as the linker region would be included. For EndoS and EndoS2, the fucosidase enzyme could be introduced by swapping out the GH18, or the fucosidase could be fused directly to the CBM domain. Additionally, different linker lengths may be applied, with variation ranging from the short GS linker to the longer G4S, (G4S) 2 and (G4S) 3.

We expect the activity to improve the most when swapping out GH18 domains for fucosidase H2 in the full length EndoE, EndoS or EndoS2, as exemplified in Fan et al. (2023 Biochemical and Biophysical Research Communications, Volume 645, Pages 40-46), for fucosidase AlfC. Next to improved activity, also expression yields are expected to increase upon fusion, as the fusion partners all express better in E. coli. Synthetic DNA encoding the proposed fusion proteins, with a N-/or C-terminal HisTag, is cloned in the E. coli expression plasmid pDEST17. Upon successful cloning and NGS verification of the sequence, the fusion proteins are transformed in BL21-AI or BL21-DE3 cells for expression. Upon overnight induction with respectively arabinose or IPTG, the bacterial cells are harvested by centrifugation (4500 g, 15′) and lysed to release the recombinant proteins. Subsequently, the proteins of interest are purified from the lysate by IMAC and SEC, and optionally IEC or HIC purification steps may be included prior to sizing, if the protein purity is suboptimal.

Next, the activity is tested on synthetic substrate (chloro-nitrophenol fucose) and on IgG or VHH-Fc, which are expressed in CHO cells, with GlcNAC₂ Man₃GlcNAc₂ as main N-glycan. To expand the number of substrates for testing the fucosidase fusions with, a pretreatment of some of the antibodies with Endo E GH20 is performed to generate GlcNAC₁Man₃GlcNAC₂ and Man₃GlcNAC₂.

Defucosylation of antibodies is tested by incubating fucosidase and antibodies overnight in several buffers (Tris, NaPi, NH4Ac) at variable pH (5.0 to 8.0). After the incubation, the N-glycans can be analyzed by DSA FACE.

Experimental procedures.

### Sequences Retrieval (Cazy Pedia, NCBI) and Phylogenetic Analysis

The sequences encoding fucosidases belonging to GH29 were extracted from the CAZy database or NCBI as described in Grootaert et al., (2020, Glycobiology, 30(9) p. 735-745). Multiple sequence alignments were performed in the Molecular Evolutionary Genetics Analysis software (version 11, MEGA11) by employing the Multiple Sequence Comparison by Log-Expectation (MUSCLE) algorithm with the standard parameters suggested by the MEGA software. Next, the best fitting model was determined and subsequently used to construct a maximum-likelihood phylogenetic tree in MEGA11. The latter step was executed 1000 times, as a bootstrap command, in order to verify the likelihood of branches clustering together.

### AlphaFold2 Model Generation

Using the sequence information for the fucosidase collection, we ran AlphaFold2 (Jumper, J., Evans, R., Pritzel, A. et al. Highly accurate protein structure prediction with AlphaFold. Nature 596, 583-589 (2021)) to predict the structure of all the candidates. Predictions were generated using AlphaFold version 2.1.1 (release date May 11, 2021). For each candidate, ten structures were predicted, as the five distinct AlphaFold models were run both on a multiple sequence alignment generated using the default JackHMMER strategy, and on one generated using the MMSeqs2 strategy as used in ColabFold (Mirdita, et al. 2022; Nat Methods 19, 679-682). Of these ten predictions, the model with highest local confidence (calculated using the average pLDDT) was selected for downstream evaluation.

Recombinant Glycosyl Hydrolase Expression in E. coli (AI and DE3) and Purification

Upon synthesizing and cloning of the encoding sequences into E. coli expression plasmids (performed Grootaert et al., 2020 (supra), E. coli BL21 strains were transformed with vectors encoding the fucosidases, either the DE3 strain for IPTG induction or Al strain for arabinose-based induction. After incubation at 37° C. to reach exponential growth, the fucosidases were expressed intracellularly for 18 h in selective LB broth at 28° C. Next, the cells were pelleted and lysed by sonication in a lysozyme containing buffer (25 mM Tris-HCl pH 7.5, 200 mM NaCl, 5 mM MgCl2, 100 μg/ml chicken egg white lysozyme and 20 mM imidazole). Upon clarification and 0.22 μm filtering of the lysate, the N-terminally HisTagged fucosidases were purified with immobilized metal-ion affinity chromatography (IMAC) on an ÄKTA chromatography system (Cytiva). Briefly, a nickel-sepharose packed column (HisTrap HP) was equilibrated with equilibration buffer (20 mM sodium phosphate, 20 mM imidazole, 0.5M NaCl, pH 7.2-8.0 depending on protein of interest's pl). After loading of the lysate, the column was washed with equilibration buffer to remove non-specifically bound contaminants. Next, all proteins were eluted with elution buffer (20 mM sodium phosphate, 300 mM imidazole, pH 7.2-8.0). Elution fractions were then analyzed by SDS-PAGE and if possible by fucosidase activity assay (see further). Fractions containing (active) protein were then pooled and concentrated over Amicon filters (with the cutoff chosen such that the molecular mass always exceeded the filter cutoff by threefold). When the IMAC elution pool was reduced to 5 ml or less, a size exclusion chromatography was performed. For all hydrolases used in this project, a Superdex200 HiLoad 16/600 (GE Healthcare), equilibrated to 20 mM Tris-HCl pH 7.5, 50 mM NaCl and 1 mM EDTA, was used for this polishing step.

For small expression volumes, gravity flow purification with NiNTA beads (17-0575-01, Cytiva) was employed to crudely purify the HisTagged proteins from the lysate. NTA beads were loaded with NiSO₄. After removing non-chelated nickel ions, the beads were mixed with E. coli lysates for 1 h at room temperature. Subsequently, the beads were collected on filter membranes (#7311550, Bio-Rad) and washed with IMAC equilibration buffer. Next, bound proteins were eluted with IMAC elution buffer.

### Quantification of Fucosidase Activity

Fucosidase activity was quantified spectrophotometrically at 415 nm on an iMark™ microplate reader (Bio-Rad) after incubation at 37° C. with chromogenic substrate 2-chloro-p-nitrophenol-fucosidase (CNP-fucose, Carbosynth) for 1 h. 50 μl of (diluted) enzyme or purification fraction was mixed with substrate, to a final concentration of 1 mM CNP-fucose. To buffer the reaction, MES pH 5.0 or sodium phosphate pH 7.0 was added to a final concentration of 50 mM. In case different pH conditions were used in the same experiment, the pH was increased by adding 1M NaOH (50% volume), in order to reduce the effect of pH on the absorbance of CNP.

VHH-Fc Production, Purification and Digest with Hexosaminidase

VHH-Fc was produced in ExpiCHO cells (Thermo Fisher Scientific) by transient transfection. Cultures were incubated at 6×10⁶ cells/ml at 37° C. and 8% CO₂. A pcDNA3.4-based expression vector for VHH-Fc was transfected using ExpiFectamine CHO reagent (0.8 μg DNA/ml culture volume). ExpiCHO enhancer and ExpiCHO feed were added, 18-22 h after transfection, according to the manufacturer's instructions. Subsequently, cells were incubated at 32° C. and 5% CO₂. A second feeding was performed 5 days post transfection. Recombinant protein expression was stopped when cell viability dropped below 75%, by pelleting the CHO cells and filtering the resulting supernatant.

VHH-Fc was purified from the supernatant using the MabSelect SuRe column (GE Healthcare) on ÄKTA chromatography systems (Cytiva). After washing with Mcllvaine buffer (phosphate/citrate buffer) pH 7.2 (Mcllvaine, 1921; Journal of Biological Chemistry 49(1) p 183-186), bound proteins were eluted in Mcllvaine pH 3. Prior to pooling and concentrating the protein-containing fractions (based on UV absorbance), the acidic pH was increased to pH 6 with saturated Na₃PO₄. For the final purification step, a Superdex200 HiLoad 16/600 (GE Healthcare), equilibrated to storage buffer (25 mM L-histidine, 125 mM NaCl pH 6), was employed.

VHH-Fc was treated with hexosaminidase to remove terminal GlcNAc residues from the N-glycan. Initially, commercially available enzyme extracted from Jack Bean (GKX-5003, Agilent) was incubated with VHH-Fc, 0.3 mU enzyme for 10 μg VHH-Fc, at 37° C. for 24 h in acidic buffer: Agilent buffer (100 mM Na-citrate phosphate pH 5.0) or NEB glycobuffer 1 (5 mM CaCl₂) and 50 mM NaAc at pH 5.5).

VHH-Fc was treated with Endo E GH20 domain, as non-commercial alternative to hexosaminidase extracted from Jack Bean. Endo E GH20 was produced in house in E. coli BL21-AI and purified as described above. Subsequently, 7.2 mg VHH-Fc was mixed with 3.6 mg Endo E GH20 in 25 ml PBS. After 24 h incubation at 37° C., the VHH-Fc was purified identically to described above (protein A and size exclusion chromatography).

### Defucosylation Screening of VHH-Fc

Experiments testing fucosidases for activity on Man₃GlcNAc₃Fuc on VHH-Fc were performed overnight (16-18 h) at 37° C. Unless specified otherwise, 2 μg VHH-Fc was treated with 2 μg fucosidase in 50 mM Na₂HPO₄ pH 7.0 in a final volume of 40-50 μl. The level of defucosylation was subsequently analyzed by DSA FACE, described below.

### DSA Face

Isolation and analysis of N-glycans was performed as described previously (Laroy et al., 2006; Nature Protocols 1, p. 397-405). Briefly, glycoproteins were denatured and immobilized on 96-well PVDF plates (Merck). After reduction and alkylation of disulfide bonds and blocking in 1% PVP, N-glycans were released by employing in-house produced PNGase F (15.4 IUB mU/ul) for 3 h at 37° C. The resulting reducing end of the anomeric oxygen was subsequently derivatized with APTS (10 mM final concentration) by reductive amination with 2-picolinoborane (2-PB). Before analysis on an ABI3130 Genetic Analyzer (Applied Biosystems) with a 50 cm capillary, excess label was removed by size exclusion chromatography over Sephadex G10 resin (GE Healthcare).

### SEQUENCE LISTING

The amino acid sequences provided herein may be presented without tags or signal peptides, though for recombinant production as described herein, the enzymes were usually N- or C-terminally HisTagged (6×His) and require a signal peptide.

1. A composition comprising:
a pretreatment enzyme, and
a GH29 fucosidase enzyme comprising SEQ ID NO: 1 or a variant or homologue with at least 85% identity to the full length thereof;
wherein the composition defucosylates an N-glycosylated Fc-containing protein.

2. The composition of claim 1, wherein the pretreatment enzyme is a hexosaminidase, or an N-glycosylated Fc-containing protein scavenger enzyme.

3. The composition of claim 2, wherein the hexosaminidase comprises SEQ ID NO: 10 or a homologue with at least 95% identity thereof, and/or the scavenger enzyme comprises any one of SEQ ID NO: 12, 14, 16 or 17, or a homologue with at least 95% identity thereof.

4. The composition of claim 1, wherein the N-glycosylated Fc-containing protein is an antibody.

5. The composition of claim 1, wherein the GH29 fucosidase and pretreatment enzyme are present in the composition as a single fusion protein.

6. The composition of claim 5, wherein the fusion protein comprises a sequence selected from the group of SEQ ID NOs: 18-21, or a homologue with at least 95% identity of any one thereof.

7. The composition of claim 1, further comprising a buffer solution.

8. The composition of claim 1, wherein the enzymes are present on a solid support.

9. (canceled)

10. The composition of claim 8, wherein the solid support is a resin, bead, plate, column, chip or membrane.

11. A method of producing a defucosylated N-glycosylated Fc-containing protein, the method comprising:
incubating a protein sample comprising a N-glycosylated Fc-containing protein with the composition of claim 1, and
isolating the defucosylated N-glycosylated Fc-containing protein.

12. The method according to claim 11, wherein the incubation is performed under physiological conditions.

13. The method according to claim 11, wherein the incubating is repeated one or more times, and wherein each repeat comprises providing fresh enzyme to the protein sample.

14. The method according to claim 11, wherein the isolating comprises removing the enzymes from the defucosylated N-glycosylated Fc-containing protein.

15. The method according to claim 11, wherein the isolating comprises eluting the defucosylated N-glycosylated Fc-containing proteins from the enzymes wherein the enzymes of the composition are present on a solid support.

16. The method according to claim 11, wherein the protein sample is a patient sample or component or fraction thereof containing IgGs.

17. A method of treating a subject in need of defucosylated IgGs, the method comprising administering to the subject defucosylated IgGs obtained by the method according to claim 16.

18. The method according to claim 11, wherein the protein sample is a sample of recombinantly produced N-glycosylated Fc-containing proteins.

19. (canceled)

20. The method according claim 16, wherein the patient sample is obtained by plasmapheresis.

21. The method according claim 17, wherein the administering comprises infusion of the defucosylated IgGs and wherein the subject is the source of the protein sample.

22. The method according to claim 18, wherein the recombinantly produced N-glycosylated Fc-containing proteins are obtained from a eukaryotic cell, a mammalian cells, or a human cell.
